(19) United States
(12) Patent Application Publication
(10) Pub. No.: US 2012/0095077 A1
(43) Pub. Date: Apr. 19, 2012

(54) METHODS AND COMPOSITIONS RELATED TO MODIFIED GUANINE BASES FOR CONTROLLING OFF-TARGET EFFECTS IN RNA INTERFERENCE

(75) Inventors: Cynthia J. Burrows, Salt Lake City, UT (US); Arunkumar Kannan, Salt Lake City, UT (US); Peter A. Beal, Davis, CA (US)

(73) Assignee: UNIVERSITY OF UTAH RESEARCH FOUNDATION, Salt Lake City, UT (US)

(21) Appl. No.: 13/257,093

(22) PCT Filed: Mar. 23, 2010
(86) PCT No.: PCT/US10/28345

§ 371 Date: Dec. 19, 2011

Related U.S. Application Data
(60) Provisional application No. 61/162,507, filed on Mar. 23, 2009.

(51) Int. Cl. A61K 31/7088; C07H 19/167; C07D 473/18; C07H 21/02

(52) U.S. Cl. 514/44 A; 536/24.5; 536/27.81; 544/276

(57) ABSTRACT

Disclosed are compositions and methods related to modified nucleobases. Also disclosed are compositions and methods related to modified interfering RNAs. Also disclosed are compositions and methods related to modified guanine bases for controlling off-target effects in RNA interference.

### CROSS-REFERENCE TO RELATED PATENT APPLICATIONS

This application claims priority to U.S. Provisional Application No. 61/162,507 filed on Mar. 23, 2009, which is hereby incorporated herein by reference in its entirety.

### STATEMENT REGARDING FEDERALLY SPONSORED RESEARCH

This invention was made with government support under NIH Grant GM 080784. The government has certain rights in this invention.

### BACKGROUND

The drug discovery process enjoyed a huge boost at the beginning of this century with the Noble Prize-winning discovery of long dsRNA (double-stranded RNA) mediated RNAi (RNA interference) in the worm (Fire et al. 1998) and the subsequent demonstration that RNAi, mediated by small-interfering RNA (siRNA), also operates in mammalian cells (Elbashir et al., 2001). New researches sprouted quickly in order to understand the RNAi mechanism and the possibility of its applications as a drug. It has been proposed that siRNA has several advantages over other available therapeutic agents (Bumcrot et al. 2006). To date at least three different siRNAs for indications such as age-related macular degeneration (AMD), a leading cause of blindness, and for respiratory syncytial virus (RSV), have completed phase I clinical trails (Michels et al. 2006, Batik et al. 2006).

siRNAs can be synthetically prepared dsRNA that can sometime range from 19-23 nucleotide long and are similar to miRNAs (micro RNAs) that are formed from long double-stranded RNA by the action of the proteins drosha and dicer. Together they can form the RISC (RNA interference silencing complex) containing Ago2 (Argonaute 2) and result in the cleavage of the targeted mRNA ultimately knocking down the expression of the desired gene (Rand et al. 2004, Ma et al. 2005, Matranga et al. 2005, Rand et al. 2005, Chiu et al. 2002) (FIG. 1).

However, when long dsRNA is injected into mammalian cells to knock down a gene, it is mostly recognized as a molecular pattern associated with viral infection. This is because many viruses have dsRNA genomes or use RNA-dependent RNA polymerases, which generate long, dsRNA products. Elbashir et al. reported that 21 bp RNA duplexes mimicking miRNAs can be added to mammalian cells and elicit potent, target-specific gene silencing and this led to the great advancement in the field of siRNA.

Despite many advantages of siRNAs, there are certain issues that need to be solved to make it a potent therapeutic agent. For example, stability of siRNAs in intracellular and extra cellular environments (Zimmermann et al. 2006, Morrissey et al. 2005, Soutschek et al. 2004), sequence independent off target effects such as binding with dsRBM proteins including PKR (RNA dependent protein kinase) and ADAR (Adenosine deaminase) (Sledz et al. 2003, Karikó et al. 2004, Yang et al. 2005), sequence dependent off target effects such as binding with genes other than target gene due to partial complementary of siRNA and other immunostimulatory effects (Hemmi et al. 2000, Judge et al. 2005, Hornung et al. 2005), and cellular permeability (Rand et al. 2005) can all be improved.

For example, soluble duplex RNA-binding proteins are potential sources of off-target effects (Sledz et al. 2006; Yang et al. 2005). Furthermore, RNA binding containing dsRBMs (double stranded RNA-binding motifs) such as PKR can interfere with the desired RNA interference effect of a siRNA duplex. (Puthenveetil et al. 2004). High resolution structures solved both by NMR and by X-ray crystallography show these motifs bind ˜16 bp of dsRNA by making contacts in two consecutive minor grooves and the opening to the intervening major groove (Ryter et al. 1998, Blaszczyk et al. 2004, Wu et al. 2004). One study showed many of the cellular proteins capable of binding a biotinylated siRNA duplex contained mainly dsRBMs, including the RNA-dependent protein kinase (PKR) (Zhang et al. 2005). Since dsRBMs bind duplex RNA by making contacts in the minor groove, they introduced a steric block at specific sites in the minor groove and analyzed the effect on PKR binding by affinity cleavage experiments (Vuyisich et al. 2002).

Targeted silencing of disease-associated genes by chemically modified siRNA holds considerable promise as a novel therapeutic strategy. However, unmodified siRNA can exhibit off-target effects.

Disclosed herein are compositions and methods for overcoming these limitations. For example, disclosed herein are compositions and methods comprising modifications of siRNA that results in a reduction or complete abrogation of these off-target effects.

### SUMMARY

In accordance with the purposes of this invention, as embodied and broadly described herein, disclosed are compositions comprising modified nucleobases, as well as methods of synthesizing and using such compositions. Also disclosed are compositions that relate to methods of blocking binding of an off-target molecule to an siRNA molecule. Also disclosed are compositions and methods comprising modifying at least one guanosine base of the siRNA molecule.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows a model for human RISC-mediated target recognition and cleavage;

FIG. 2 shows the structures of sugar, backbone and base modifications and of the cholesterol conjugate;

FIG. 3 shows protein binding sites on duplex RNA can be blocked by site-selective steric occlusion of the minor groove. Benzylation of guanosine 6 in a G:U wobble pair found in stem-loop IV of EBER-1 blocks binding by dsRBM I of PKR;

FIG. 4 shows (A) siRNA duplex designed to knockdown expression of human caspase 2. Shown are 5′-GGAAAUGCAAGAGAAACUGTT-3′ (SEQ ID NO: 1) and 3′-dGTCCUUUACGUUCUCUUUGAC-5′ (SEQ ID NO: 2). (B) N²-benzyl modification of nucleotides near positions 7, 9 and 14 of the sense strand blocked binding to the four dsRBMs identified. (C) siRNA duplexes bearing such modifications show dose-dependent knockdown of caspase 2 mRNA in HeLa cells, indicating that the N²-benzyl substitutions facing the minor groove prevents its interaction with dsRBM containing proteins while maintaining the ability to knockdown gene expression (Concentrations of siRNA tested=10, 30 and 100 nM);

FIGS. 5A and 5B show a model for the function of RNAi alkylated purine switches with N²-alkylated 8-oxoG. Watson-Crick pairing in the siRNA duplex projects steric bulk into the minor groove to inhibit the binding of dsRBMs in off-target proteins. Hoogsteen pairing of 8-oxoG (syn) with A (anti) in the target mRNA hides the steric bulk in the deep major groove of A-form RNA;

FIG. 6 shows preliminary caspase2 knock down studies;

FIG. 7 shows a scheme for synthesis of N²-alkyl-8-oxodG-phosphoramidite;

FIG. 8 shows caspase 2 knock down studies-dual luciferase assay for (A) the propyl series and (B) the benzyl series of siRNA modifications;

FIG. 9 shows a strategy for blocking sequence-specific off target effects by modified bases. (A) OdG-U rich immunostimulatory siRNAs interact with TLR 7 likely via base specific recognition. (B) Alkylated OdG probably change the shapes of bases and prevent interaction with receptors like TLR 7;

FIG. 10 shows siRNA (small interfering RNA) and its mechanism. 19-25 nucleotide long double-stranded RNA molecules exogenously (artificially) introduced into cells by various transfection methods to bring about the specific knockdown of a gene of interest;

FIG. 11 shows N²-alkyl-8-oxo-dG Phosphoramidite and anti-sense strand of caspase-2 (5′-CAGXUUCUCUXGCAUXUCCtt-3′ (SEQ ID NO: 15));

FIG. 12 shows an assay system—psiCHECK-2 vector;

FIG. 13 shows a plasmid preparation;

FIG. 14 shows caspase-2 gene knockdown assay using luminenscence;

FIG. 15 shows T_{M}.studies of singly modified interfering siRNAs;

FIG. 16 shows T_{M}.studies of doubly and triply modified interfering siRNAs; and

FIG. 17 shows the results of the PKR binding studies described in Example 4

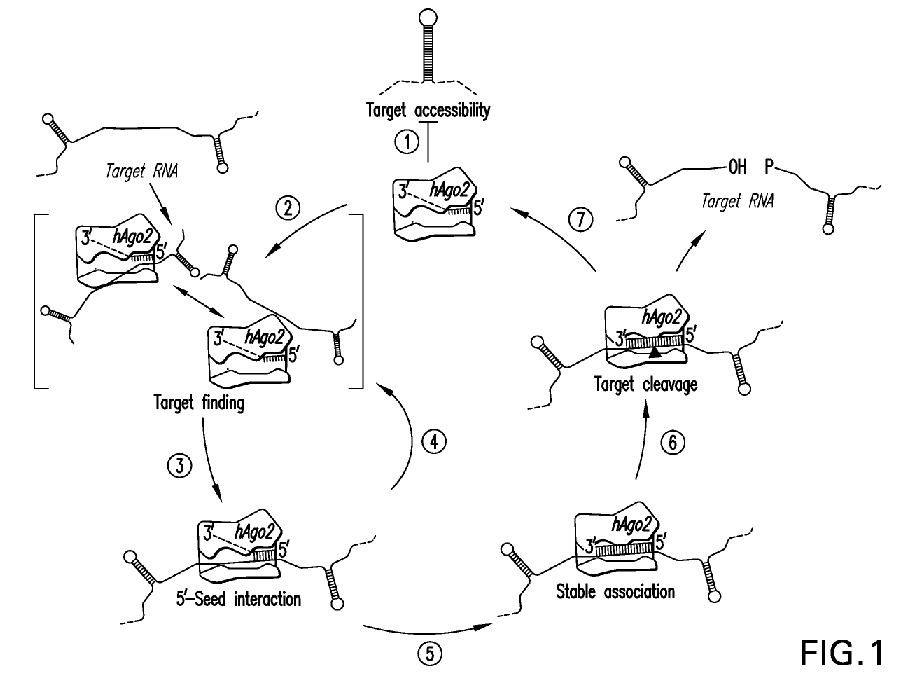

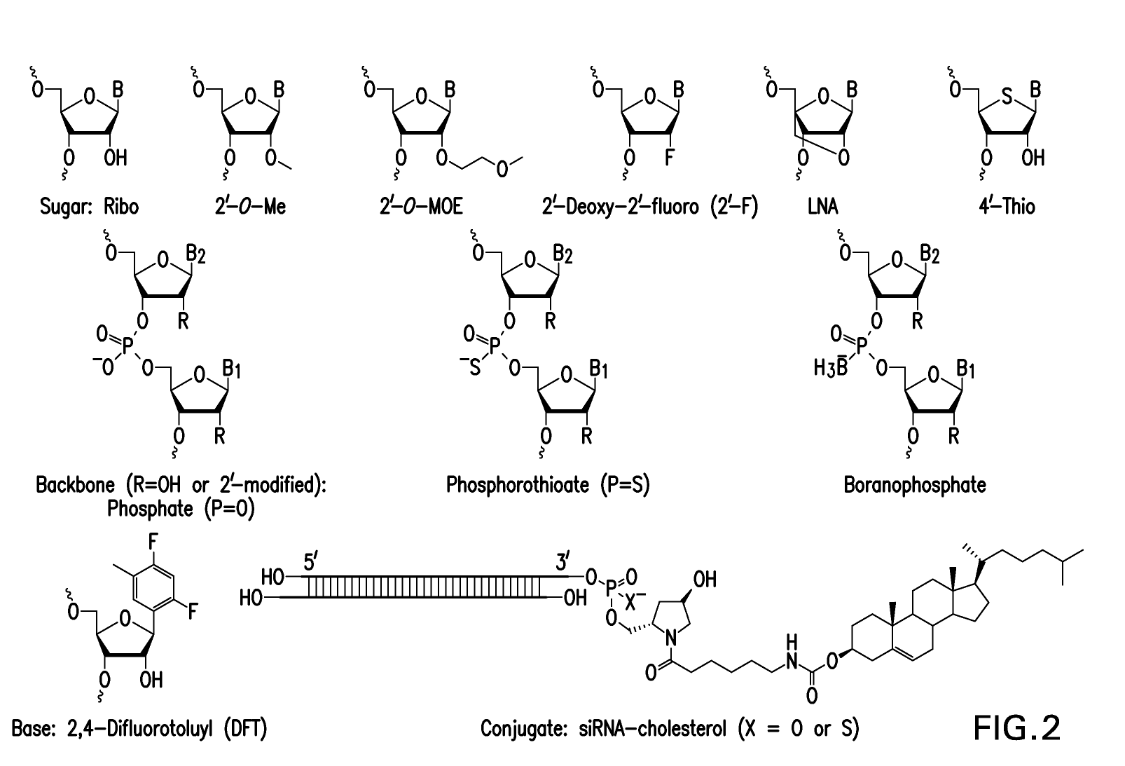

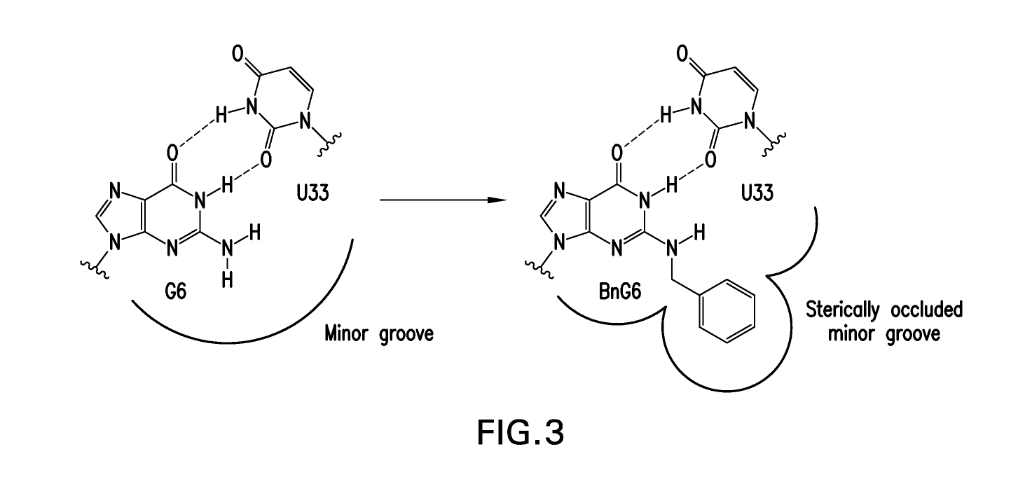

### DETAILED DESCRIPTION

Before the present compounds, compositions, articles, devices, and methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

A. Definitions

specification and in the claims which follow, reference will be made to a number of terms which shall be defined to have the following meanings:

As used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

The term “interfering RNA” or “RNAi” or “interfering RNA sequence” refers to double-stranded RNA (i.e., duplex RNA) that is capable of reducing or inhibiting expression of a target gene (i.e., by mediating the degradation of mRNAs which are complementary to the sequence of the interfering RNA) when the interfering RNA is in the same cell as the target gene. Interfering RNA thus refers to the double stranded RNA formed by two complementary strands or by a single, self-complementary strand. Interfering RNA may have substantial or complete identity to the target gene or may comprise a region of mismatch (i.e., a mismatch motif). The sequence of the interfering RNA can correspond to the full length target gene, or a subsequence thereof.

Interfering RNA includes “short interfering RNA,” “siRNA,” “short interfering nucleic acid,” “antisense RNA” or “siRNA,” e.g., interfering RNA of about 15-60, 15-50, or 15-40 (duplex) nucleotides in length, more typically about, 15-30, 15-25 or 19-25 (duplex) nucleotides in length, and is preferably about 20-24, 21-22, or 21-23 (duplex) nucleotides in length (e.g., each complementary sequence of the double stranded siRNA is 15-60, 15-50, 15-40, 15-30, 15-25, or 19-25 nucleotides in length, preferably about 20-24, 21-22, or 21-23 nucleotides in length, and the double-stranded siRNA is about 15-60, 15-50, 15-40, 15-30, 15-25, or 19-25 base pairs in length, preferably about 20-24, 21-22, or 21-23 base pairs in length). siRNA duplexes may comprise 3′ overhangs of about 1 to about 4 nucleotides or about 2 to about 3 nucleotides and 5′ phosphate termini. Examples of siRNA include, without limitation, a double-stranded polynucleotide molecules assembled from two separate oligonucleotides, wherein one strand is the sense strand and the other is the complementary antisense strand; a double-stranded polynucleotide molecule assembled from a single oligonucleotide, where the sense and antisense regions are linked by a nucleic acid-based or non-nucleic acid-based linker; a double-stranded polynucleotide molecule with a hairpin secondary structure having self-complementary sense and antisense regions; and a circular single-stranded polynucleotide molecule with two or more loop structures and a stem having self-complementary sense and antisense regions, where the circular polynucleotide can be processed in vivo or in vitro to generate an active double-stranded siRNA molecule.

“Modified interfering RNA” refers to interfering RNA that comprises at least one modified nucleoside described herein, e.g., modified guanosine. Modified interfering RNA targeting can mediate potent silencing of the target sequence. Modified interfering RNA can reduce or completely abrogate the off-target response to interfering RNA.

“Modified nucleoside”, “modified nucleotide”, or “modified base” refers to a nucleoside or nucleotide comprising an alteration, change in chemical structure, or addition to a purine ring. For example, a “modified nucleoside”, “modified nucleotide” or “modified base” can refer to a compound comprising formula (III) or formula (VI), as well as the additional embodiments of the formulas, as described herein. A “modified nucleoside”, “modified nucleotide” or “modified base” can refer to a “modified guanosine” or “modified guanosine base” wherein the guanosine comprises formula (III) or formula (VI), as well as the additional embodiments of the formulas described herein. The modified nucleosides (e.g., modified guanosine) disclosed herein can also be used with interfering RNA. Interfering RNA can be designed to interact with a target nucleic acid molecule through either canonical or non-canonical base pairing. A target nucleic acid molecule can be any nucleic acid. For example a “target nucleic acid molecule” can be DNA, RNA, cDNA, mRNA, or a DNA/RNA hybrid. A target molecule can be a protein or gene of interest.

A “gene of interest” or “sequence of interest” can include one or more transcriptional regulatory sequences and any other nucleic acid, such as introns, that may be necessary for optimal expression of a selected or target nucleic acid. The term “gene of interest” or “sequence of interest” can mean a nucleic acid sequence (e.g., a therapeutic gene), that is partly or entirely heterologous, i.e., foreign, to a cell into which it is introduced. The term “gene of interest” or “sequence of interest” can also mean a nucleic acid sequence, that is partly or entirely homologous to an endogenous gene of the cell into which it is introduced, but which is designed to be inserted into the genome of the cell in such a way as to alter the genome (e.g., it is inserted at a location which differs from that of the natural gene or its insertion results in “a knockout”). The term “gene of interest” or “sequence of interest” can also mean a nucleic acid sequence, that is partly or entirely complementary to an endogenous gene of the cell into which it is introduced. A “protein of interest” means a peptide or polypeptide sequence (e.g., a therapeutic protein), that is expressed from a sequence of interest or gene of interest.

The interaction of the interfering RNA and the target molecule is designed to promote the destruction of the target molecule through, for example, RNAseH mediated RNA-DNA hybrid degradation. Alternatively the interfering RNA is designed to interrupt a processing function that normally would take place on the target molecule, such as transcription or replication. Interfering RNA can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that interfering RNAs bind the target molecule with a dissociation constant (kd) less than or equal to 10⁻⁶, 10⁻⁸, 10⁻¹⁰, or 10⁻¹². A representative sample of methods and techniques which aid in the design and use of interfering RNAs can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

siRNA can be chemically synthesized. siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al. 2002; Calegari et al. 2002; Byrom et al. 2003; Kawasaki et al. 2003; Knight and Bass 2001; and Robertson et al. 1968). Preferably, dsRNA are at least 50 nucleotides to about 100, 200, 300, 400, or 500 nucleotides in length. A dsRNA may be as long as 1000, 1500, 2000, 5000 nucleotides in length, or longer. The dsRNA can encode for an entire gene transcript or a partial gene transcript. In certain instances, siRNA may be encoded by a plasmid (e.g., transcribed as sequences that automatically fold into duplexes with hairpin loops).

As used herein, the term “mismatch motif” or “mismatch region” refers to a portion of an siRNA sequence that does not have 100% complementarity to its target sequence. An siRNA may have at least one, two, three, four, five, six, or more mismatch regions. The mismatch regions may be contiguous or may be separated by 1, 2, 3, 4, 5, 6, 7, 8, 9, 10, 11, 12, or more nucleotides. The mismatch motifs or regions may comprise a single nucleotide or may comprise two, three, four, five, or more nucleotides.

An “effective amount” or “therapeutically effective amount” of an siRNA is an amount sufficient to produce the desired effect, e.g., an inhibition of expression of a target sequence in comparison to the normal expression level detected in the absence of the siRNA Inhibition of expression of a target gene or target sequence is achieved when the value obtained with the siRNA relative to the control is about 90%, 80%, 70%, 60%, 50%, 40%, 30%, 25%, 20%, 15%, 10%, 5%, or 0%. Suitable assays for measuring expression of a target gene or target sequence include, e.g. examination of protein or mRNA levels using techniques known to those of skill in the art such as dot blots, northern blots, in situ hybridization, ELISA, immunoprecipitation, enzyme function, as well as phenotypic assays known to those of skill in the art.

As used herein, the term “responder cell” refers to a cell, for example a mammalian cell, that produces a detectable response when contacted with an siRNA.

“Substantial identity” refers to a sequence that hybridizes to a reference sequence under stringent hybridization conditions, or to a sequence that has a specified percent identity over a specified region of a reference sequence.

The phrase “stringent hybridization conditions” refers to conditions under which an siRNA will hybridize to its target sequence, typically in a complex mixture of nucleic acids, but to no other sequences. Stringent hybridization conditions are sequence-dependent and will be different in different circumstances. Longer sequences hybridize specifically at higher temperatures. An extensive guide to the hybridization of nucleic acids is found in Tijssen 1993. Generally, stringent hybridization conditions are selected to be about 5-10° C. lower than the thermal melting point for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at T_{M}, 50% of the probes are occupied at equilibrium). Stringent hybridization conditions may also be achieved with the addition of destabilizing agents such as formamide. For selective or specific hybridization, a positive signal is at least two times background, preferably 10 times background hybridization.

Exemplary stringent hybridization conditions can be as follows: 50% formamide, 5×SSC, and 1% SDS, incubating at 42° C., or 5×SSC, 1% SDS, incubating at 65° C., with wash in 0.2×SSC, and 0.1% SDS at 65° C. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. 1990.

Nucleic acids that do not hybridize to each other under stringent hybridization conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, Ausubel et al, eds.

The terms “substantially identical” or “substantial identity,” in the context of two or more nucleic acids, refer to two or more sequences or subsequences that are the same or have a specified percentage of nucleotides that are the same (i.e., at least about 60%, preferably at least about 65%, 70%, 75%, 80%, 85%, 90%, or 95% identity over a specified region), when compared and aligned for maximum correspondence over a comparison window, or designated region as measured using one of the following sequence comparison algorithms or by manual alignment and visual inspection. This definition, when the context indicates, also refers analogously to the complement of a sequence. Preferably, the substantial identity exists over a region that is at least about 5, 10, 15, 20, 25, 30, 35, 40, 45, 50, 75, or 100 nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of a number of contiguous positions selected from the group consisting of from about 20 to about 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman 1981, by the homology alignment algorithm of Needleman and Wunsch 1970, by the search for similarity method of Pearson and Lipman 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology, Ausubel et al., eds. (1995 supplement)).

An example of algorithms that are suitable for determining percent sequence identity and sequence similarity are the BLAST and BLAST 2.0 algorithms, which are described in Altschul et al. 1977 and Altschul et al.1990, respectively. BLAST and BLAST 2.0 are used, with the parameters described herein, to determine percent sequence identity for the nucleic acids and proteins of the invention. Software for performing BLAST analyses is publicly available through the National Center for Biotechnology Information.

The BLAST algorithm also performs a statistical analysis of the similarity between two sequences (see, e.g., Karlin and Altschul 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The term “nucleic acid” or “polynucleotide” refers to a polymer containing at least two deoxyribonucleotides or ribonucleotides in either single- or double-stranded form and include DNA and RNA. DNA may be in the form of, e.g., antisense oligonucleotides, plasmid DNA, pre-condensed DNA, a PCR product, vectors (P1, PAC, BAC, YAC, artificial chromosomes), expression cassettes, chimeric sequences, chromosomal DNA, or derivatives and combinations of these groups. RNA may be in the form of siRNA, mRNA, tRNA, rRNA, tRNA, vRNA, and combinations thereof. Nucleic acids include nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such modifications are disclosed herein.

The term “gene” refers to a nucleic acid (e.g., DNA or RNA) sequence that comprises partial length or entire length coding sequences necessary for the production of a polypeptide or precursor polypeptide.

“Gene product,” as used herein, refers to a product of a gene such as an RNA transcript or a polypeptide.

“Systemic delivery,” as used herein, refers to delivery that leads to a broad biodistribution of a compound such as an siRNA within an organism. Some techniques of administration can lead to the systemic delivery of certain compounds, but not others. Systemic delivery means that a useful, preferably therapeutic, amount of a compound is exposed to most parts of the body. To obtain broad biodistribution generally requires a blood lifetime such that the compound is not rapidly degraded or cleared (such as by first pass organs (liver, lung, etc.) or by rapid, nonspecific cell binding) before reaching a disease site distal to the site of administration. Systemic delivery can be by any means known in the art including, for example, intravenous, subcutaneous, and intraperitoneal.

“Local delivery,” as used herein, refers to delivery of a compound such as an siRNA directly to a target site within an organism. For example, a compound can be locally delivered by direct injection into a disease site such as a tumor or other target site such as a site of inflammation or a target organ such as the liver, heart, pancreas, kidney, and the like.

The term “mammal” refers to any mammalian species such as a human, mouse, rat, dog, cat, hamster, guinea pig, livestock, and the like. For example, a mammal can be a human.

As described herein, a “subject” can be an animal, e.g., a human being or a mammal. A subject can also be a non-human animal. Examples of a non-human animal include but are not limited to a mouse, rat, pig, monkey, chimpanzee, orangutan, cat, dog, sheep, and cow. A subject can be a natural animal. A subject can also be a transgenic, non-human animal including but not limited to a transgenic mouse or transgenic rat.

By “sample” is meant an animal; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic 15 acid), which is assayed as described herein. A sample may also be any body fluid or excretion (for example, but not limited to, blood, urine, stool, saliva, tears, bile) that contains cells or cell components.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

B. Compositions

Disclosed herein are compounds of Formula I:

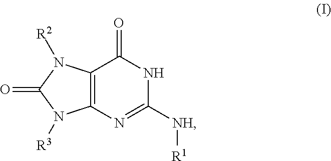

wherein R1 can be: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; (v) substituted or unsubstituted C₁-C₉ heteroaryl; or (vi) substituted or unsubstituted C₁-C₉ heterocyclic; provided that R¹ does not comprise pyrenyl, 1-oxopropyl, or tetrahydrofuranyl; wherein R² can be: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; (v) substituted or unsubstituted C₁-C₉ heteroaryl; (vi) substituted or unsubstituted C₁-C₉ heterocyclic; or (vii) hydrogen; wherein R³ can be: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; (v) substituted or unsubstituted C₁-C₉ heteroaryl; (vi) substituted or unsubstituted C₁-C₉ heterocyclic; or (vii) hydrogen.

As used herein, “alkyl” refers to a chemical substituent having at least one saturated carbon atom. The alkyl substituents can be linear, branched, or cyclic alkyl. Examples of C₁-C₆ linear or branched alkyl include without limitation methyl (C₁), ethyl (C₂), n-propyl (C₃), iso-propyl (C₃), n-butyl (C₄), sec-butyl (C₄), iso-butyl (C₄), tert-butyl (C₄), pentyl (C₅), iso-pentyl (C₅), hexyl (C₆). The linear or branched alkyl can be substituted or unsubsituted with a variety of substituents, including halogen, hydroxyl, carboxy, amino, amido, cyano, thio, among others. Specific examples of substituted linear or branched include without limitation hydroxymethyl (C₁), chloromethyl (C₁), trifluoromethyl (C₁), aminomethyl (C₁), 1-chloroethyl (C₂), 2-hydroxyethyl (C₂), 1,2-difluoroethyl (C₂), 3-carboxypropyl (C₃), and the like.

Cyclic alkyl groups can comprise rings having from 3 to 20 carbon atoms, wherein the atoms which comprise said rings are limited to carbon atoms, and further each ring can be independently substituted with one or more moieties capable of replacing one or more hydrogen atoms. The following are non-limiting examples of substituted and unsubstituted cyclic alkyl groups which encompass the following categories of units: cyclic rings having a single substituted or unsubstituted hydrocarbon ring, non-limiting examples of which include, cyclopropyl (C₃), 2-methyl-cyclopropyl (C₃), cyclopropenyl (C₃), cyclobutyl (C₄), 2,3-dihydroxycyclobutyl (C₄), cyclobutenyl (C₄), cyclopentyl (C₅), cyclopentenyl (C₅), cyclopentadienyl (C₅), cyclohexyl (C₆), cyclohexenyl (C₆), cycloheptyl (C₇), cyclooctanyl (C₈), decalinyl (C₁₀), 2,5-dimethylcyclopentyl (C₅), 3,5-dichlorocyclohexyl (C₆), 4-hydroxycyclohexyl (C₆), and 3,3,5-trimethylcyclohex-1-yl (C₆); cyclic rings having two or more substituted or unsubstituted fused hydrocarbon rings, non-limiting examples of which include, octahydropentalenyl (C₈), octahydro-1H-indenyl (C₉), 3a,4,5,6,7,7a-hexahydro-3H-inden-4-yl (C₉), decahydroazulenyl (C₁₀); cyclic rings which are substituted or unsubstituted bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

As used herein, “alkenyl” refers to a chemical substituent having one or more —C═C— double bonds. The alkenyl substituent can be linear, branched, or cyclic alkenyl. Examples of which include without limitation ethenyl (C₂), 3-propenyl (C₃), 1-propenyl (also 2-methylethenyl) (C₃), isopropenyl (also 2-methylethen-2-yl) (C₃), buten-4-yl (C₄), and the like; substituted linear or branched alkenyl, non-limiting examples of which include, 2-chloroethenyl (also 2-chlorovinyl) (C₂), 4-hydroxybuten-1-yl (C₄), 7-hydroxy-7-methyloct-4-en-2-yl (C₉), 7-hydroxy-7-methyloct-3,5-dien-2-yl (C₉), and the like.

The term “alkynyl” as used herein refers to a substituents having at least one carbon-carbon triple bond and includes linear, branched, and cyclic alkynyl, non-limiting examples of which include, ethynyl (C₂), prop-2-ynyl (also propargyl) (C₃), propyn-1-yl (C₃), and 2-methyl-hex-4-yn-1-yl (C₇); substituted linear or branched alkynyl, non-limiting examples of which include, 5-hydroxy-5-methylhex-3-ynyl (C₇), 6-hydroxy-6-methylhept-3-yn-2-yl (C₈), 5-hydroxy-5-ethylhept-3-ynyl (C₉), and the like.

Any of the alkyl, alkenyl, or alkyl groups defined above can also comprise heteroatoms within a carbon chain, including for example, O, S, N, or combinations thereof. Thus, ethers, secondary amines, and thiols can be present in any of the above defined groups. Thus, as defined herein, “alkyl” includes groups such as “alkoxy,” including for example, methoxy.

The term “aryl” as used herein refers to a chemical units encompassing at least one phenyl or naphthyl ring and further each ring can be independently substituted with one or more moieties capable of replacing one or more hydrogen atoms.” The following are non-limiting examples of “substituted and unsubstituted aryl rings” which encompass the following categories of units: C₆ or C₁₀ substituted or unsubstituted aryl rings; phenyl and naphthyl rings whether substituted or unsubstituted, non-limiting examples of which include, phenyl (C₆), naphthylen-1-yl (C₁₀), naphthylen-2-yl (C₁₀), 4-fluorophenyl (C₆), 2-hydroxyphenyl (C₆), 3-methylphenyl (C₆), 2-amino-4-fluorophenyl (C₆), 2-(N,N-diethylamino)phenyl (C₆), 2-cyanophenyl (C₆), 2,6-di-tert-butylphenyl (C₆), 3-methoxyphenyl (C₆), 8-hydroxynaphthylen-2-yl (C₁₀), 4,5-dimethoxynaphthylen-1-yl (C₁₀), and 6-cyano-naphthylen-1-yl (C₁₀); C₆ or C₁₀ aryl rings fused with 1 or 2 saturated rings non-limiting examples of which include, bicyclo[4.2.0]octa-1,3,5-trienyl (C₈), and indanyl (C₉).

The term “heteroaryl” as used herein includes those units encompassing one or more rings comprising from 5 to 20 atoms wherein at least one atom in at least one ring is a heteroatom chosen from nitrogen (N), oxygen (O), or sulfur (S), or mixtures of N, O, and S, and wherein further at least one of the rings which comprises a heteroatom is an aromatic ring. The following are non-limiting examples of “substituted and unsubstituted heterocyclic rings” which encompass the following categories of units: heteroaryl rings containing a single ring, non-limiting examples of which include, 1,2,3,4-tetrazolyl (C₁), [1,2,3]triazolyl (C₂), [1,2,4]triazolyl (C₂), triazinyl (C₃), thiazolyl (C₃), 1H-imidazol (C3), oxazolyl (C₃), furanyl (C₄), thiopheneyl (C₄), pyrimidinyl (C₄), 2-phenylpyrimidinyl (C₄), pyridinyl (C₅), 3-methylpyridinyl (C₅), and 4-dimethylaminopyridinyl (C₅) heteroaryl rings containing 2 or more fused rings one of which is a heteroaryl ring, non-limiting examples of which include: 7H-purinyl (C₅), 9H-purinyl (C₅), 6-amino-9H-purinyl (C₅), 5H-pyrrolo[3,2-d]pyrimidinyl (C₆), 7H-pyrrolo[2,3-d]pyrimidinyl (C₆), pyrido[2,3-d]pyrimidinyl (C₇), 2-phenylbenzo[d]thiazolyl (C₇), 1H-indolyl (C₈), 4,5,6,7-tetrahydro-1-H-indolyl (C₈), quinoxalinyl (C₈), 5-methylquinoxalinyl (C₈), quinazolinyl (C₈), quinolinyl (C₉), 8-hydroxy-quinolinyl (C₉), and isoquinolinyl (C₉).

The terms “heterocyclic” and/or “heterocycle” as used herein refer to those units comprising one or more rings having from 3 to 20 atoms wherein at least one atom in at least one ring is a heteroatom chosen from nitrogen (N), oxygen (O), or sulfur (S), or mixtures of N, O, and S, and wherein further the ring which comprises the heteroatom is also not an aromatic ring. The following are non-limiting examples of “substituted and unsubstituted heterocyclic rings” which encompass the following categories of units: heterocyclic units having a single ring containing one or more heteroatoms, non-limiting examples of which include, diazirinyl (C₁), aziridinyl (C₂), urazolyl (C₂), azetidinyl (C₃), pyrazolidinyl (C₃), imidazolidinyl (C₃), oxazolidinyl (C₃), isoxazolinyl (C₃), isoxazolyl (C₃), thiazolidinyl (C₃), isothiazolyl (C₃), isothiazolinyl (C₃), oxathiazolidinonyl (C₃), oxazolidinonyl (C₃), hydantoinyl (C₃), tetrahydrofuranyl (C₄), pyrrolidinyl (C₄), morpholinyl (C₄), piperazinyl (C₄), piperidinyl (C₄), dihydropyranyl (C₅), tetrahydropyranyl (C₅), piperidin-2-onyl (valerolactam) (C₅), 2,3,4,5-tetrahydro-1H-azepinyl (C₆), 2,3-dihydro-1H-indole (C₈), and 1,2,3,4-tetrahydro-quinoline (C₉); heterocyclic units having 2 or more rings one of which is a heterocyclic ring, non-limiting examples of which include hexahydro-1H-pyrrolizinyl (C₇), 3a,4,5,6,7,7a-hexahydro-1H-benzo[d]imidazolyl (C₇), 3a,4,5,6,7,7a-hexahydro-1H-indolyl (C₈), 1,2,3,4-tetrahydroquinolinyl (C₉), and decahydro-1H-cycloocta[b]pyrrolyl (C₁₀).

The term “halogen” is intended to refer to Br, Cl, I, and F.

The term “amino” refers to any substituted or unsubstituted primary, secondary, or tertiary amine.

The term “substituted” is used throughout the specification. The term “substituted” is applied to the units described herein as a substituted unit or moiety which has one or more hydrogen atoms replaced by a substituent or several substituents as defined herein below. The units, when substituting for hydrogen atoms are capable of replacing one hydrogen atom, two hydrogen atoms, or three hydrogen atoms of a hydrocarbyl moiety at a time. In addition, these substituents can replace two hydrogen atoms on two adjacent carbons to form said substituent, new moiety, or unit. For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. Three hydrogen replacement includes cyano, and the like. The term substituted is used throughout the present specification to indicate that a hydrocarbyl moiety, inter alia, aromatic ring, alkyl chain; can have one or more of the hydrogen atoms replaced by a substituent. When a moiety is described as “substituted” any number of the hydrogen atoms may be replaced. For example, 4-hydroxyphenyl is a “substituted aromatic carbocyclic ring (aryl ring)”, (N,N-dimethyl-5-amino)octanyl is a “ substituted C₈ linear alkyl unit, 3-guanidinopropyl is a “substituted C₃ linear alkyl unit,” and 2-carboxypyridinyl is a “substituted heteroaryl unit.”

The following are non-limiting examples of units which can be substituents on a residue or chemical moiety that is defined as substituted: i) C₁-C₁₂ linear, branched, or cyclic alkyl, alkenyl, and alkynyl; methyl (C₁), ethyl (C₂), ethenyl (C₂), ethynyl (C₂), n-propyl (C₃), iso-propyl (C₃), cyclopropyl (C₃), 3-propenyl (C₃), 1-propenyl (also 2-methylethenyl) (C₃), isopropenyl (also 2-methylethen-2-yl) (C₃), prop-2-ynyl (also propargyl) (C₃), propyn-1-yl (C₃), n-butyl (C₄), sec-butyl (C₄), iso-butyl (C₄), tert-butyl (C₄), cyclobutyl (C₄), buten-4-yl (C₄), cyclopentyl (C₅), cyclohexyl (C₆); ii) substituted or unsubstituted C₆ or C₁₀ aryl; for example, phenyl, naphthyl (also referred to herein as naphthylen-1-yl (C₁₀) or naphthylen-2-yl (C₁₀)); iii) substituted or unsubstituted C₆ or C₁₀ alkylenearyl; for example, benzyl, 2-phenylethyl, naphthylen-2-ylmethyl; iv) substituted or unsubstituted C₁-C₉ heterocyclic rings; as described herein; v) substituted or unsubstituted C₁-C₉ heteroaryl rings; as described herein; vi) —(CR^{102a}R^{102b})ₐOR¹⁰¹; for example, —OH, —CH₂OH, —OCH₃, —CH₂OCH₃, —OCH₂CH₃, —CH₂OCH₂CH₃, —OCH₂CH₂CH₃, and —CH₂OCH₂CH₂CH₃; vii) —(CR^{102a}R^{102b})ₐC(O)R¹⁰¹; for example, —COCH₃, —CH₂COCH₃, —OCH₂CH₃, —CH₂COCH₂CH₃, —COCH₂CH₂CH₃, and —CH₂COCH₂CH₂CH₃; vii) —(CR^{102a}R^{102b})ₐC(O)OR¹⁰¹; for example, —CO₂CH₃, —CH₂CO₂CH₃, —CO₂CH₂CH₃, —CH₂CO₂CH₂CH₃, —CO₂CH₂CH₂CH₃, and —CH₂CO₂CH₂CH₂CH₃; —(CR^{102a}R^{102b})ₐC(O)N(R¹⁰¹)₂; for example, —CONH₂, —CH₂CONH₂, —CONHCH₃, —CH₂CONHCH₃, —CON(CH₃)₂, and —CH₂CON(CH₃)₂;—(CR^{102a}R^{102b})ₐN(R¹⁰¹)₂; for example, —NH₂, —CH₂NH₂, —NHCH₃, —CH₂NHCH₃, —N(CH₃)₂, and —CH₂N(CH₃)₂; halogen; —F, —Cl, —Br, and —I; —(CR^{102a}R^{102b})ₐCN;—(CR^{102a}R^{102b})ₐNO₂; —CHⱼXₖ; wherein X is halogen, the index j is an integer from 0 to 2, j+k=3; for example, —CH₂F, —CHF₂, —CF₃, —CCl₃, or —CBr₃; (CR^{102a}R^{102b})^{a}SR¹⁰¹; —SH, —CH₂SH, —SCH₃, —CH₂SCH₃, —SC₆H₅, and —CH₂SC₆H₅; —(CR^{120a}R^{102b})ₐSO₂R¹⁰¹; for example, —SO₂H, —CH₂SO₂H, —SO₂CH₃,—CH₂SO₂CH₃, —SO₂C₆H₅, and —CH₂SO₂C₆H₅; and —(CR^{102a}R^{102b})ₐSO₃R¹⁰¹; for example, —SO₃H, —CH₂SO₃H, —SO₃CH₃, —CH₂SO₃CH₃, —SO₃C₆H₅, and —CH₂SO₃C₆H₅; wherein each R¹⁰¹ is independently hydrogen, substituted or unsubstituted C₁-C₄ linear, branched, or cyclic alkyl, phenyl, benzyl, heterocyclic, or heteroaryl; or two R¹⁰¹ units can be taken together to form a ring comprising 3-7 atoms; R^{102a} and R^{102b} are each independently hydrogen or C₁-C₄ linear or branched alkyl; the index “a” is from 0 to 4.

Also disclosed herein are compounds of Formula I: wherein R¹ is substituted or unsubstituted methyl, ethyl, n-propyl, iso-propyl, n-butyl, iso-butyl, sec-butyl, tent-butyl, or benzyl. Also disclosed herein are compounds of Formula I: wherein R² is hydrogen.

Also disclosed herein are compounds of Formula I: wherein R³ is substituted or unsubstituted tetrahydrofuranyl or tetrahydropyranyl. Also disclosed herein are compounds of Formula I: wherein R³ is a residue of Formula II:

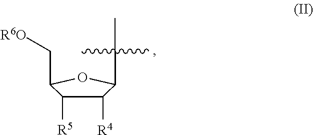

wherein R⁴ can be: (i) hydrogen; (ii) hydroxyl; (iii) alkoxy; (iv) amino; or (v) halogen; wherein R⁵ can be: (i) hydrogen; (ii) hydroxyl; (iii) alkoxy; (iv) amino; (v) halogen; (vi) C₁-C₁₂ phosphonite, phosphate, phosphonate, or phosphoryl; or (vii) an O-linked solid support; and wherein R⁶ is: (i) hydrogen; (ii) a protecting group; (iii) a monophosphate; (iv) a diphosphate; (v) a triphosphate; (vi) a nucleotide; or (vii) a deoxynucleotide.

Also disclosed herein are compounds of Formula I: wherein R³ is a residue of Formula II:

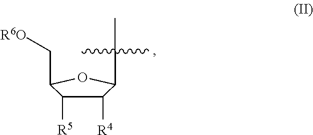

wherein R⁴ can be: (i) hydrogen; (ii) hydroxyl; (iii) alkoxy; (iv) amino; or (v) halogen; wherein R⁵ is: (i) —O—(N,N-diisopropyl O-methyl phosphoramidite) or —O—(N,N-diisopropyl O-2-cyanoethyl phosphoramidite); and wherein R⁶ is: (i) hydrogen; (ii) a protecting group; (iii) a monophosphate; (iv) a diphosphate; (v) a triphosphate; (vi) a nucleotide; or (vii) a deoxynucleotide

Also disclosed herein are compounds of Formula I: wherein R³ is a residue of Formula II:

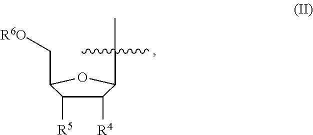

wherein R⁴ can be: (i) hydrogen; (ii) hydroxyl; (iii) alkoxy; (iv) amino; or (v) halogen; wherein R⁵ can be: (i) hydrogen; (ii) hydroxyl; (iii) alkoxy; (iv) amino; (v) halogen; (vi) C₁-C₁₂ phosphonite, phosphate, phosphonate, or phosphoryl; or (vii) an O-linked solid support; and wherein R⁶ is: (i) dimethoxytrityl (DMT); (ii) monomethoxytrityl; (iii) 9-phenylxanthen-9-yl (Pixyl); or (iv) 9-(p-methoxyphenyl)xanthen-9-yl (Mox).

Also disclosed herein are nucleosides of Formula III:

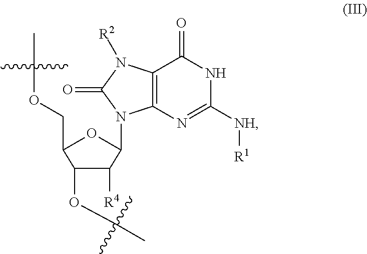

wherein R¹ can be: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; (v) substituted or unsubstituted C₁-C₉ heteroaryl; or (vi) substituted or unsubstituted C₁-C₉ heterocyclic; provided that R¹ does not comprise pyrenyl, 1-oxopropyl, or tetrahydrofuranyl; wherein R² can be: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; (v) substituted or unsubstituted C₁-C₉ heteroaryl; (vi) substituted or unsubstituted C₁-C₉ heterocyclic; or (vii) hydrogen; and wherein R⁴ can be: (i) hydrogen; (ii) hydroxyl; (iii) alkoxy; (vi) amino; or (v) halogen.

Also disclosed herein are siRNA molecules comprising at least one modified guanosine. For example, disclosed herein are siRNA molecules comprising a compound comprising Formula I:

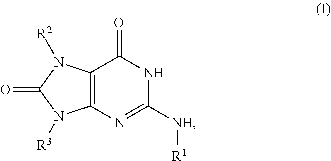

wherein R1 can be: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; (v) substituted or unsubstituted C₁-C₉ heteroaryl; or (vi) substituted or unsubstituted C₁-C₉ heterocyclic; provided that R¹ does not comprise pyrenyl, 1-oxopropyl, or tetrahydrofuranyl; wherein R² can be: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; (v) substituted or unsubstituted C₁-C₉ heteroaryl; (vi) substituted or unsubstituted C₁-C₉ heterocyclic; or (vii) hydrogen; wherein R³ can be: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; (v) substituted or unsubstituted C₁-C₉ heteroaryl; (vi) substituted or unsubstituted C₁-C₉ heterocyclic; or (vii) hydrogen.

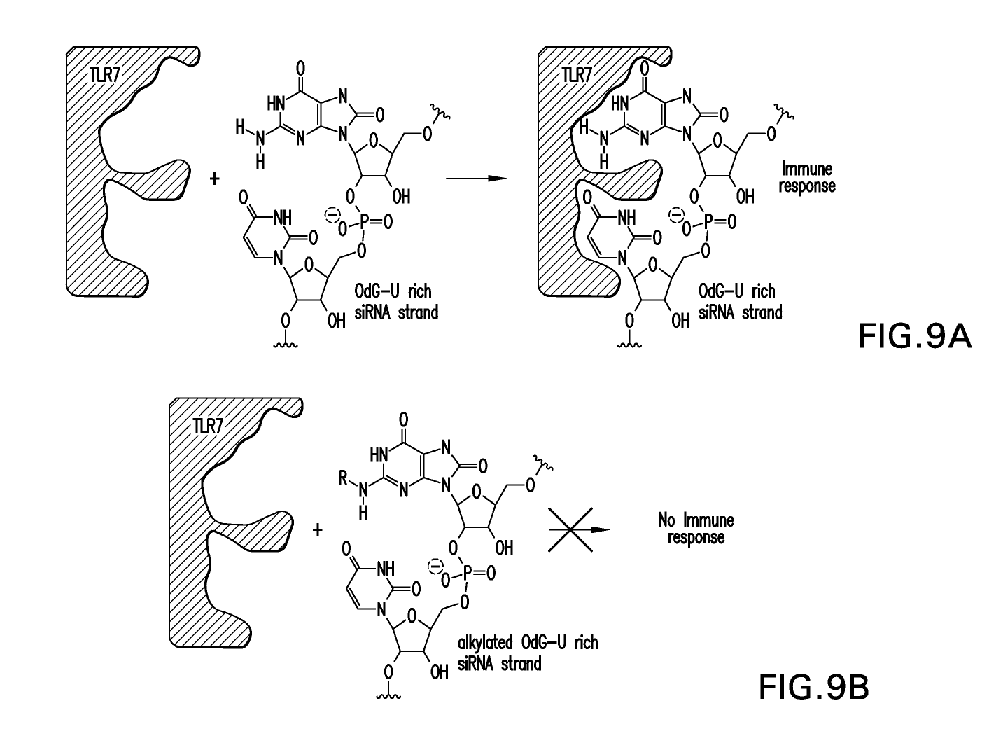

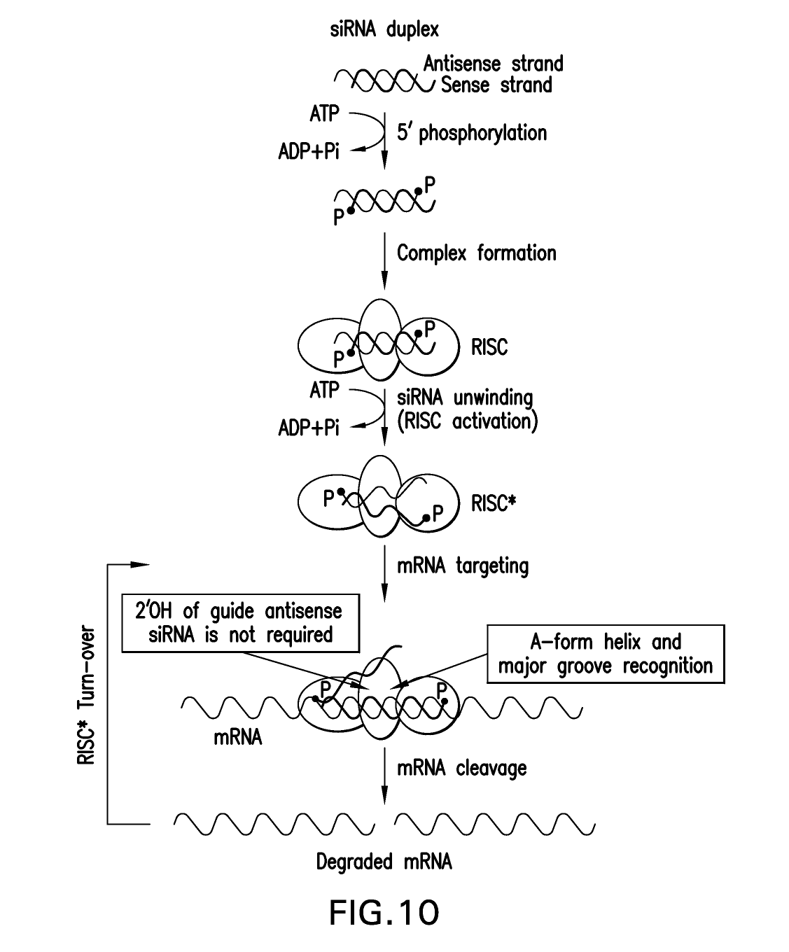

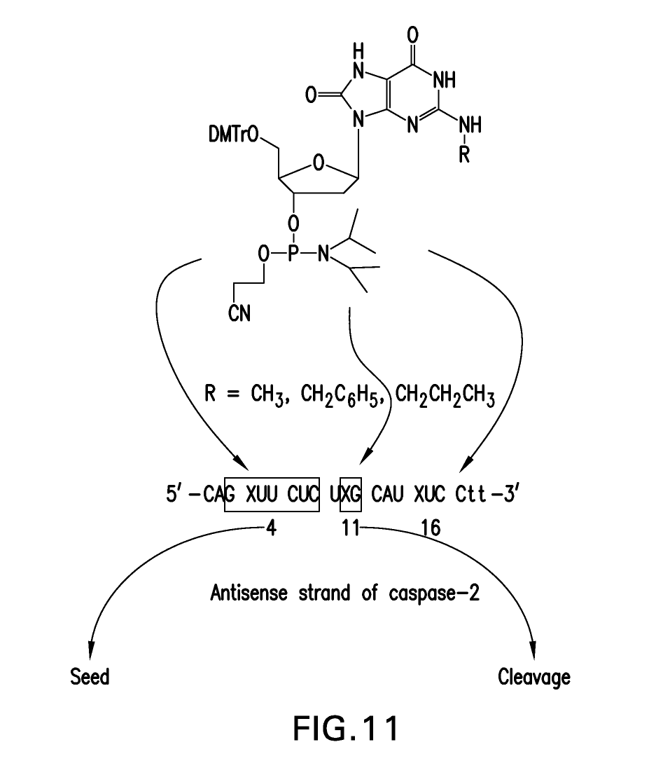

Generally, R¹ can comprise any suitable group that would sterically hinder the binding of the nucleobase with a cellular double-stranded RNA-binding protein. With reference to FIG. 9, for example, R¹ (labeled R in FIG. 9) of an exemplary OdG-U rich siRNA strand can effectively inhibit the binding of the OdG-U rich siRNA strand with the Toll-like receptor 7 (TLR7) immune gene, thereby avoiding an undesirable immune response in a subject that has been administered the OdG-U rich siRNA strand. In specific embodiments, R¹ is substituted or unsubstituted methyl, ethyl, n-propyl, iso-propyl, n-butyl, iso-butyl, sec-butyl, tent-butyl, or benzyl.

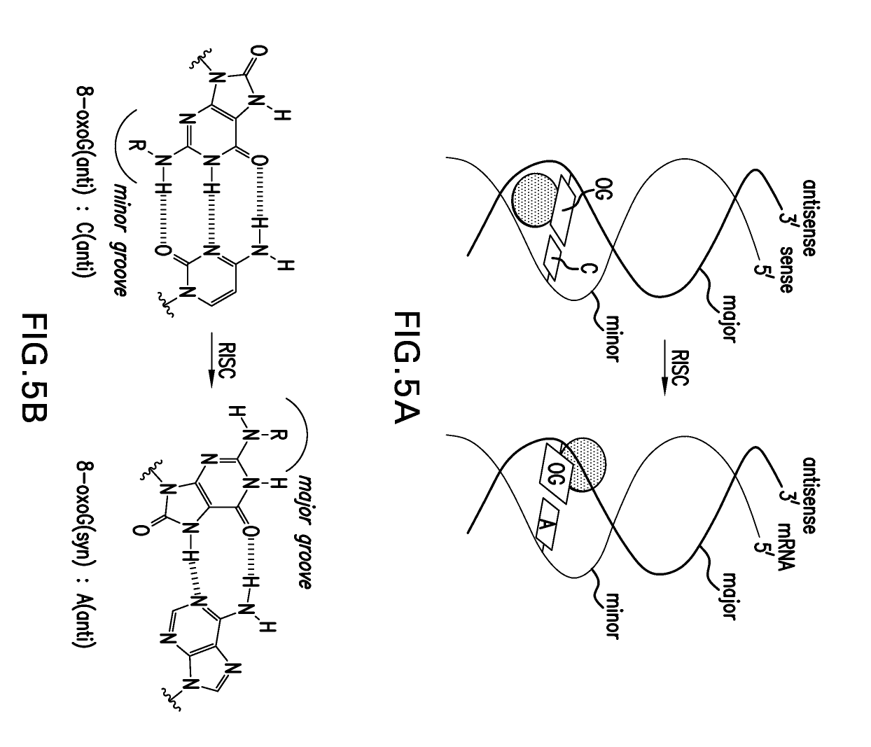

The substituent R² can comprise a variety of groups, depending on the desired mode of action of the nucleobase. With reference to FIG. 5, an exemplary nucleobase can bind in the minor groove of RNA with C in a typical Watson-Crick pairing. In this example, the substituent at R² is not involved in the pairing and can thus be any of those groups defined above. However, again with reference to FIG. 5, a Hoogsten pairing between the nucleobase of the invention and A involves the substituent at R² as a hydrogen bond donor. Thus, in this example, R² is preferably hydrogen.

The substituent R³ can generally comprise any suitable group, but typically comprises a cyclic group. Specific examples include without limitation substituted or unsubstituted tetrahydrofuranyl or tetrahydropyranyl. In one embodiment, R³ is represented by the formula:

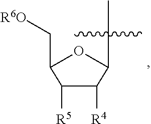

wherein R⁴ is i) hydrogen; ii) hydroxyl; iii) alkoxy; iv) amino; or v) halogen; R⁵ is: i) hydrogen; ii) hydroxyl; iii) alkoxy; iv) amino; or v) halogen; vi) C₁-C₁₂phosphonite, phosphate, phosphonate, or phosphoryl; vii) an O-linked solid support; and R⁶ is: i) hydrogen; ii) a protecting group; or iii) a nucleoside; or iv) a deoxynucleoside. In various embodiments, the nucleobase can be in oxyribose or deoxyribose form, and as such R⁴ can be hydroxyl, alkoxy, protected hydroxyl, or hydrogen.

When R⁵ comprises a C₁-C₁₂ phosphonite, phosphate, phosphonate, or phosphoryl group, phosphonite, phosphate, phosphonate, or phosphoryl group can be protected with a suitable protecting group. Protecting groups for such residues are attached to the phosphorus-bound oxygen, and serve to protect the phosphorus during oligonucleotide synthesis. See, for example, Oligonucleotides and Analogues: A Practical Approach, Eckstein, F., Ed., IRL Press, Oxford, U.K. 1991, which is incorporated herein by this reference, for its teachings of phosphonite, phosphate, phosphonate, and phosphoryl protecting groups. One exemplary phosphoryl protecting group is the cyanoethyl group. Other exemplary phosphoryl protecting groups include 4-cyano-2-butenyl groups, methyl groups, and diphenylmethylsilylethyl (DPSE) groups. In one specific embodiment, R⁵ can comprise —O—(N,N-diisopropyl O-methyl phosphoramidite) or —O—(N,N-diisopropyl O-2-cyanoethyl phosphoramidite). These two groups, for example, are suitable for use when incorporating the nucleobase into a nucleic acid strand, such as RNA.

When the nucleobase is present in a strand of a nucleic acid, R⁵ can be hydroxyl if the nucleobase terminates the strand, or R⁵ can be a suitable nucleoside. When R⁵ is hydroxyl, it can be protected. Thus, in various embodiments, a disclosed nucleic acid strand, such as a strand of RNA, can comprise a structural residue represented by the formula:

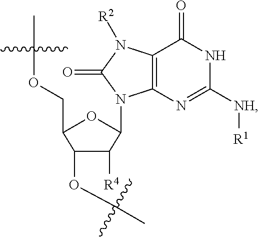

wherein R¹ is: i) substituted or unsubstituted C₁-C₆ linear, branched, or cyclic alkyl; ii) substituted or unsubstituted C₂-C₆ linear, branched, or cyclic alkenyl; iii) substituted or unsubstituted C₂-C₆ linear or branched alkynyl; iv) substituted or unsubstituted C₆-C₁₀ aryl; v) substituted or unsubstituted C₁-C₉ heteroaryl; vi) substituted or unsubstituted C₁-C₉ heterocyclic; provided that R¹ does not comprise pyrenyl, 1-oxopropyl, or tetrahydrofuranyl; R² is: i) substituted or unsubstituted C₁-C₆ linear, branched, or cyclic alkyl; ii) substituted or unsubstituted C₂-C₆ linear, branched, or cyclic alkenyl; iii) substituted or unsubstituted C₂-C₆ linear or branched alkynyl; iv) substituted or unsubstituted C₆-C₁₀ aryl; v) substituted or unsubstituted C₁-C₉ heteroaryl; vi) substituted or unsubstituted C₁-C₉ heterocyclic; or vii) hydrogen; and R⁴ is: i) hydrogen; or ii) hydroxyl.

Also disclosed herein are siRNA molecules comprising at least one modified guanosine, wherein the base opposite the modified guanosine is not complementary. Also disclosed herein are siRNA molecules comprising at least one modified guanosine, wherein the efficacy of the siRNA molecule is increased.

Also disclosed herein are methods for making an alkylated compound, comprising, alkylating the amino group at position 6 of a compound of Formula IV,

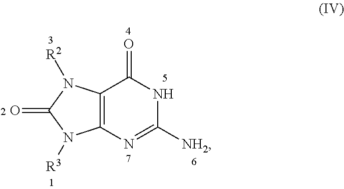

resulting in an alkylated compound of Formula V:

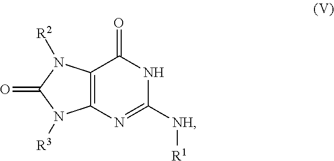

wherein R¹ can be: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; (v) substituted or unsubstituted C₁-C₉ heteroaryl; or (vi) substituted or unsubstituted C₁-C₉ heterocyclic; provided that R¹ does not comprise pyrenyl, 1-oxopropyl, or tetrahydrofuranyl; wherein R² can be: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; (v) substituted or unsubstituted C₁-C₉ heteroaryl; (vi) substituted or unsubstituted C₁-C₉ heterocyclic; or (vii) hydrogen; and wherein R³ can be: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; (v) substituted or unsubstituted C₁-C₉ heteroaryl; (vi) substituted or unsubstituted C₁-C₉ heterocyclic; or (vii) hydrogen.

Also disclosed herein are methods for making an alkylated compound, comprising, alkylating the amino group at position 6 of a compound of Formula IV, wherein alkylating the amino group comprises reacting the compound of Formula IV with an aldehyde of formula R¹CHO, wherein R¹ can be: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; (v) substituted or unsubstituted C₁-C₉ heteroaryl; or (vi) substituted or unsubstituted C₁-C₉ heterocyclic; provided that R¹ does not comprise pyrenyl, 1-oxopropyl, or tetrahydrofuranyl.

Also disclosed herein are methods for making an alkylated compound, comprising, alkylating the amino group at position 6 of a compound of Formula IV, wherein alkylating the amino group comprises reacting the compound of Formula IV with a compound of formula R¹X, wherein R¹ can be: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; (v) substituted or unsubstituted C₁-C₉ heteroaryl; or (vi) substituted or unsubstituted C₁-C₉ heterocyclic; provided that R¹ does not comprise pyrenyl, 1-oxopropyl, or tetrahydrofuranyl; and X is Br, I, F, or Cl.

Alkylating the amino group at the 6 position of the compound of step a can comprise c) reacting the compound of step a with a compound represented by the formula R1CHO, wherein R1 is: i) substituted or unsubstituted C1-C6 linear, branched, or cyclic alkyl; ii) substituted or unsubstituted C2-C6 linear, branched, or cyclic alkenyl; iii) substituted or unsubstituted C2-C6 linear or branched alkynyl; iv) substituted or unsubstituted C6-C10 aryl; v) substituted or unsubstituted C1-C9 heteroaryl; or vi) substituted or unsubstituted C1-C9 heterocyclic; provided that R1 does not comprise pyrenyl, 1-oxopropyl, or tetrahydrofuranyl; and d) reducing the product of step b to provide the alkylated nucleobase.

Alkylating the amino group at the 6 position of the compound of step a) can comprise reacting the compound of step a with a compound represented by the formula R1X, wherein R1 is: i) substituted or unsubstituted C1-C6 linear, branched, or cyclic alkyl; ii) substituted or unsubstituted C2-C6 linear, branched, or cyclic alkenyl; iii) substituted or unsubstituted C2-C6 linear or branched alkynyl; iv) substituted or unsubstituted C6-C10 aryl; v) substituted or unsubstituted C1-C9 heteroaryl; or vi) substituted or unsubstituted C1-C9 heterocyclic; provided that R1 does not comprise pyrenyl, 1-oxopropyl, or tetrahydrofuranyl; and X is Br, I, F, or Cl.

Also disclosed herein are oligonucleotides or polynucleotides comprising at least one of Formula VI:

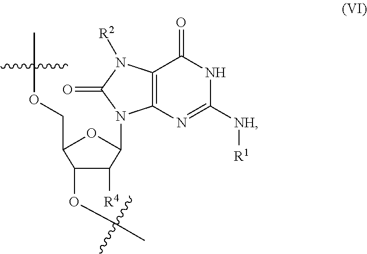

wherein R¹ can be: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; (v) substituted or unsubstituted C₁-C₉ heteroaryl; or (vi) substituted or unsubstituted C₁-C₉ heterocyclic; provided that R¹ does not comprise pyrenyl, 1-oxopropyl, or tetrahydrofuranyl; wherein R² can be: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; (v) substituted or unsubstituted C₁-C₉ heteroaryl; (vi) substituted or unsubstituted C₁-C₉ heterocyclic; or (vii) hydrogen; and wherein R⁴ can be: (i) hydrogen; (ii) hydroxyl; (iii) alkoxy; (iv) amino; or (v) halogen.

Disclosed herein are modified nucleobases such as modified guanosisnes that can be incorporated into either strand of an siRNA duplex and can reduce or completely abrogate the off-target response to synthetic interfering RNA. For example, disclosed herein are compositions comprising modified nucleobases, such as modified guanosisnes, on the sense strand of a double-stranded nucleic acid molecule. Also disclosed herein are compositions comprising modified nucleobases, such as modified guanosisnes, on the anti-sense strand of a double-stranded nucleic acid molecule.

As described herein modified antisense RNA targeting can mediate potent silencing of its target molecule such as mRNA. The approach to antisense RNA design and delivery described herein is widely applicable and advances synthetic antisense RNA into a broad range of therapeutic areas. For example, disclosed herein is a method of synthesizing 2′-deoxy-N²-alkyl-7,8-dihydro-8-oxoguanosines as cyanoethylphosphoramidites wherein “alkyl” is n-propyl or benzyl or, for the purposes of comparison, hydrogen and wherein many other alkyl groups can be envisioned by the same synthetic route. The modified guanosines, X, are individually incorporated into synthetic RNA oligonucleotides at one or more positions in which a single X:C base pair replaces a U:A base pair in the antisense:sense duplex.

Disclosed herein are compositions comprising chemically modified antisense RNA molecules and methods of using such antisense RNA to silence target gene expression.

As disclosed herein, N²-alkyl-8-oxodG (FIG. 5) can be used as a switch (existing in syn as well as anti forms) that can form Watson-Crick pairing with C in the sense strand and later Hoogsten pairing with A in mRNA as part of the RISC. As shown in FIG. 5, the alkyl group at N² is used as a steric blockade in the minor groove of RNA in a way that maintains hydrogen-bonded base pairs in an A-form duplex. In the delivery form, the steric blockade prevents non-productive binding to cellular double-stranded RNA-binding proteins. As the siRNA reaches the RISC and is unwound by a helicase, the alkylated 8-oxoguanosine undergoes a conformational change to the syn form, and it now becomes complementary to an adenosine in the mRNA target. The presence of an oxo group at C8 of purines can increase the propensity of the purine to flip from the normal anti conformation to syn, where it exposes the Hoogsteen face of the purine to base-pairing (Ames et al. 1993, Wang et al. 1998). This makes 8-oxoG(syn) accept A as its complement (FIG. 5). This can result in the switching of the N²-alkyl chain, the steric blockade, from the minor groove (anti) to the major groove (syn) of duplex RNA. Situated in the major groove, the alkyl group is buried in a deep pocket and it is unlikely to interfere with protein binding. Disclosed herein are examples demonstrating that siRNA comprising modified nucleobases can be used as a switch and form Watson-Crick (anti) pairing and Hoogsten pairing (anti) while binding with the sense strand and that mRNA was not compromised.

Disclosed herein are methods of synthesizing a series of N²-alkyl-8-oxo-2′-deoxyguanosines and methods of incorporating the modified nucleobases into various positions within siRNAs. Also disclosed are methods of determining the effects of antisense RNAs comprising modified nuclobases on RNA interference, including the off-target effects of such siRNAs. Also disclosed are methods and compositions for increasing the efficacy of antisense RNA by reducing the off-target effects.

Disclosed herein are antisense RNAs capable of silencing expression of a target sequence. The antisense RNA can comprise from about 18 to about 38 nucleotides. For example, disclosed are antisense RNAs that comprise from about 15 to about 30 nucleotides.

Disclosed herein are antisense RNAs comprising at least one modified guanosine, as described herein. The modified guanosine can be present in one strand (i.e., sense or antisense) or both strands of the siRNA. The antisense RNA sequences can have overhangs (e.g., 3′ or 5′ overhangs as described in Elbashir et al. 2001 or Nykanen et al. 2001, or may lack overhangs (i.e., have blunt ends).

According to the methods described herein, antisense RNA can be modified to decrease their off-target interactions without having a negative impact on RNAi activity. For example, a modified interfering RNA can be capable of silencing expression of the target sequence. This can lead to increased siRNA activity.

Suitable antisense RNA sequences can be identified using any means known in the art. Typically, the methods described in Elbashir et al. 2001 and Elbashir et al. 2001 can be combined with rational design rules set forth in Reynolds et al. 2004.

Generally, the sequence within about 50 to about 100 nucleotides 3′ of the AUG start codon of a transcript from the target gene of interest is scanned for dinucleotide sequences (e.g., AA, CC, GG, or UU) (see, e.g., Elbashir et al. 2001). The nucleotides immediately 3′ to the dinucleotide sequences are identified as potential interfering RNA target sequences. Typically, the 19, 21, 23, 25, 27, 29, 31, 33, 35, or more nucleotides immediately 3′ to the dinucleotide sequences are identified as potential siRNA target sites. In some embodiments, the dinucleotide sequence is an AA sequence and the 19 nucleotides immediately 3′ to the AA dinucleotide are identified as a potential siRNA target site. Interfering RNA target sites can be spaced at different positions along the length of the target gene. To further enhance silencing efficiency of the interfering RNA sequences, potential interfering RNA target sites may be further analyzed to identify sites that do not contain regions of homology to other coding sequences. For example, a suitable interfering RNA target site of about 21 base pairs typically will not have more than 16-17 contiguous base pairs of homology to other coding sequences. If the interfering RNA sequences are to be expressed from an RNA Pol III promoter, interfering RNA target sequences lacking more than 4 contiguous A's or T's are selected.

Once the potential interfering RNA target site has been identified, interfering RNA sequences complementary to the interfering RNA target sites may be designed. To enhance their silencing efficiency, the interfering RNA sequences may also be analyzed by a rational design algorithm to identify sequences that have one or more of the following features: (1) G/C content of about 25% to about 60% G/C; (2) at least 3 A/Us at positions 15-19 of the sense strand; (3) no internal repeats; (4) an A at position 19 of the sense strand; (5) an A at position 3 of the sense strand; (6) a U at position 10 of the sense strand; (7) no G/C at position 19 of the sense strand; and (8) no G at position 13 of the sense strand. Interfering RNA design tools that incorporate algorithms that assign suitable values of each of these features and are useful for selection of interfering RNA can be found at Ambion Technical Bulletin No. 506 (http://www.ambion.com/techlib/tb/tb_{—}506.html) and Yuan et al., 2004. Interfering RNA can be provided in several forms including, e.g., as one or more isolated small-interfering RNA (siRNA) duplexes, as longer double-stranded RNA (dsRNA), or as siRNA or dsRNA transcribed from a transcriptional cassette in a DNA plasmid. The siRNA sequences may have overhangs (e.g., 3′ or 5′ overhangs as described in Elbashir et al. 2001 or Nykanen et al. 2001, or may lack overhangs (i.e., to have blunt ends).

An RNA population can be used to provide long precursor RNAs, or long precursor RNAs that have substantial or complete identity to a selected target sequence can be used to make the interfering RNA. The RNAs can be isolated from cells or tissue, synthesized, and/or cloned according to methods well known to those of skill in the art. The RNA can be a mixed population (obtained from cells or tissue, transcribed from cDNA, subtracted, selected, etc.), or can represent a single target sequence. RNA can be naturally occurring (e.g., isolated from tissue or cell samples), synthesized in vitro (e.g., using T7 or SP6 polymerase and PCR products or a cloned cDNA), or chemically synthesized.

To form a long dsRNA, for synthetic RNAs, the complement can also be transcribed in vitro and hybridized to form a dsRNA. If a naturally occurring RNA population is used, the RNA complements are also provided (e.g., to form dsRNA for digestion by E. coli RNAse III or Dicer), e.g., by transcribing cDNAs corresponding to the RNA population, or by using RNA polymerases. The precursor RNAs can then hybridized to form double stranded RNAs for digestion. The dsRNAs can be directly administered to a subject or can be digested in vitro prior to administration.

Methods for isolating RNA, synthesizing RNA, hybridizing nucleic acids, making and screening cDNA libraries, and performing PCR are well known in the art (see, e.g., Gubler and Hoffman, Gene, 25:263-269 (1983); Sambrook et al., supra; Ausubel et al., supra), as are PCR methods (see, U.S. Pat. Nos. 4,683,195 and 4,683,202; PCR Protocols: A Guide to Methods and Applications (Innis et al., eds, 1990)). Expression libraries are also well known to those of skill in the art. Additional basic texts disclosing the general methods of use in this invention include Sambrook et al., Molecular Cloning, A Laboratory Manual (2nd ed. 1989); Kriegler, Gene Transfer and Expression. A Laboratory Manual (1990); and Current Protocols in Molecular Biology (Ausubel et al., eds., 1994).

Disclosed herein are methods of blocking binding of an off-target molecule to an interfering RNA molecule, the method comprising modifying at least one guanosine base of the interfering RNA molecule. The interfering RNA can comprise two or more modified guanosine bases. Examples of modified bases are found below. The off-target molecule can be any double stranded RNA-binding motif (dsRBM). For example, the off-target molecule can be PKR or ADAR. The off-target molecule can also be Toll-Like Receptor-7 (TLR-7).

The term “blocking” refers to inhibiting the interaction between siRNA and an off-target molecule. For example, the interaction between an off-target molecule and the modified interfering siRNA can be inhibited or reduced by 10, 20, 30, 40, 50, 60, 70, 80, 90, or 100%, or any amount in between.

By “off-target molecule” is meant a molecule other than the target intended to interact with the siRNA molecule. This can be any molecule at all that may come into contact with the siRNA that is not the intended target.

The nucleobases of the invention can be made using a variety of methods. A suitable precursor to the nucleobases is a compound represented by the formula:

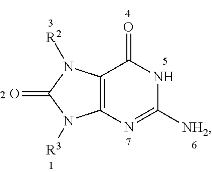

wherein each R² and R³ is independently: i) substituted or unsubstituted C₁-C₆ linear, branched, or cyclic alkyl; ii) substituted or unsubstituted C₂-C₆ linear, branched, or cyclic alkenyl; iii) substituted or unsubstituted C₂-C₆ linear or branched alkynyl; iv) substituted or unsubstituted C₆-C₁₀ aryl; v) substituted or unsubstituted C₁-C₉ heteroaryl; vi) substituted or unsubstituted C₁-C₉ heterocyclic; or vii) hydrogen. Such a precursor can be provided using a variety of methods. To form the carbonyl of the imidazole ring, an imidazole precursor can be oxidized. In one specific embodiment, the precursor to the nucleobase can be provided according to Scheme 1.

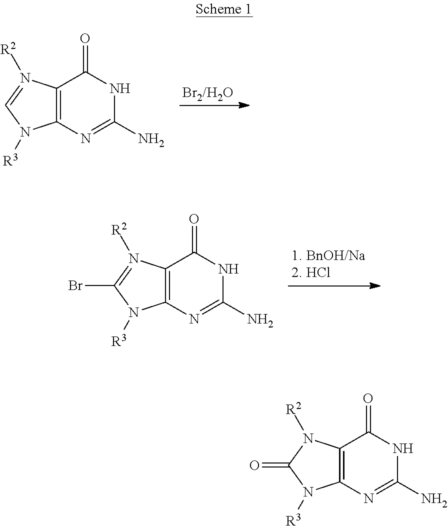

The “6” amino position of the precursor shown above can then be alkylated to provide the desired nucleobase. A variety of alkylation protocols can be used, for example, which generally utilize an electrophilic compound that can react with the nucleophilic “6” amino group.

In one specific embodiment, alkylating the amino group at the 6 position of the precursor compound comprises: c) reacting the precursor compound with a compound represented by the formula R¹CHO, wherein R¹ is: i) substituted or unsubstituted C₁-C₆ linear, branched, or cyclic alkyl; ii) substituted or unsubstituted C₂-C₆ linear, branched, or cyclic alkenyl; iii) substituted or unsubstituted C₂-C₆ linear or branched alkynyl; iv) substituted or unsubstituted C₆-C₁₀ aryl; v) substituted or unsubstituted C₁-C₉ heteroaryl; or vi) substituted or unsubstituted C₁-C₉ heterocyclic; provided that R¹ does not comprise pyrenyl, 1-oxopropyl, or tetrahydrofuranyl; and d) reducing the product of step b to provide the alkylated nucleobase.

In another specific embodiment, alkylating the amino group at the 6 position of precursor compound comprises reacting the precursor compound with a compound represented by the formula R¹X, wherein R¹ is: i) substituted or unsubstituted C₁-C₆ linear, branched, or cyclic alkyl; ii) substituted or unsubstituted C₂-C₆ linear, branched, or cyclic alkenyl; iii) substituted or unsubstituted C₂-C₆ linear or branched alkynyl; iv) substituted or unsubstituted C₆-C₁₀ aryl; v) substituted or unsubstituted C₁-C₉ heteroaryl; or vi) substituted or unsubstituted C₁-C₉ heterocyclic; provided that R¹ does not comprise pyrenyl, 1-oxopropyl, or tetrahydrofuranyl; and X is Br, I, F, or Cl.

Thus, in the above described embodiment for making the nucleobase, the final compound can be provided according to the general Scheme 2.

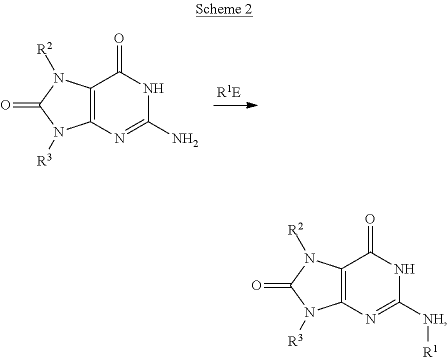

wherein E is an electrophile.

The nucleobases of the invention can be incorporated into a nucleic acid strand using methods known in the art. To incorporate the nucleobase into a nucleic acid strand, R³ will typically be a cyclic moiety, such as a sugar moiety, as discussed above which has attached thereto a nucleic acid coupling agent. Numerous examples are known in the art, including phosphodiesters, phosphotriesters, phosphate trimesters, phosphonates, phosphoramidites, among others. For a detailed explanation of how to incorporate the nucleobases into a nucleic acid strand, see Blackburn and Williams 2006, which is incorporated herein by this reference for its teaching of methods for incorporating nucleobases into nucleic acid strands. When incorporating the disclosed nucleobases in strands of nucleic acids, it can be useful to protect vulnerable groups, for example hydroxyl groups with a suitable protecting group.

Thus, it certain embodiments, it can be desirable to protect R⁴ when R⁴ is present as a hydroxyl group. Likewise, when R⁶ is present, R⁶ can comprise a suitable protecting group as desired. A wide variety of hydroxyl protecting groups can be used. Representative hydroxyl protecting groups are disclosed by Beaucage et al. 1992, and also in e.g., Green and Wuts 1991, both of which are incorporated herein by this reference, for their teachings of hydroxyl protecting groups. Specific examples of hydroxyl protecting include dimethoxytrityl (DMT), monomethoxytrityl, 9-phenylxanthen-9-yl (Pixyl) and 9-(p-methoxyphenyl)xanthen-9-yl (Mox). Other examples include various silyl ethers, such as tert-butyl dimethyl silyl either (TBDMS).

The protecting groups can be removed as desired, for example after the nucleobase has been incorporated into a strand of DNA or RNA. The R⁶ or R⁴ protecting group, when present, for example, can be removed by techniques well known in the art to form the free hydroxyl group. For example, dimethoxytrityl (DMT) protecting groups can be removed by protic acids such as formic acid, dichloroacetic acid, trichloroacetic acid, p-toluene sulphonic acid or with a Lewis acids such as zinc bromide.

The modified interfering RNA molecules of the present invention can be synthesized via a tandem synthesis technique, wherein both strands are synthesized as a single continuous oligonucleotide fragment or strand separated by a cleavable linker that is subsequently cleaved to provide separate fragments or strands that hybridize to form the interfering RNA duplex. The linker can be a polynucleotide linker or a non-nucleotide linker. The tandem synthesis of modified interfering RNA can be readily adapted to both multiwell/multiplate synthesis platforms as well as large scale synthesis platforms employing batch reactors, synthesis columns, and the like. Alternatively, the modified interfering RNA molecules of the present invention can be assembled from two distinct oligonucleotides, wherein one oligonucleotide comprises the sense strand and the other comprises the antisense strand of the interfering RNA. For example, each strand can be synthesized separately and joined together by hybridization or ligation following synthesis and/or deprotection. In certain other instances, the modified interfering RNA molecules of the present invention can be synthesized as a single continuous oligonucleotide fragment, where the self-complementary sense and antisense regions hybridize to form an interfering RNA duplex having hairpin secondary structure.

In certain embodiments, in addition to the modified guanosines, the interfering RNA molecules of the present invention further comprise one or more chemical modifications such as terminal cap moieties, phosphate backbone modifications, and the like. Examples of terminal cap moieties include, without limitation, inverted deoxy abasic residues, glyceryl modifications, 4′,5′-methylene nucleotides, 1-(β-D-erythrofuranosyl) nucleotides, 4′-thio nucleotides, carbocyclic nucleotides, 1,5-anhydrohexitol nucleotides, L-nucleotides, α-nucleotides, modified base nucleotides, threo-pentofuranosyl nucleotides, acyclic 3′,4′-seco nucleotides, acyclic 3,4-dihydroxybutyl nucleotides, acyclic 3,5-dihydroxypentyl nucleotides, 3′-3′-inverted nucleotide moieties, 3′-3′-inverted abasic moieties, 3′-2′-inverted nucleotide moieties, 3′-2′-inverted abasic moieties, 5′-5′-inverted nucleotide moieties, 5′-5′-inverted abasic moieties, 3′-5′-inverted deoxy abasic moieties, 5′-amino-alkyl phosphate, 1,3-diamino-2-propyl phosphate, 3-aminopropyl phosphate, 6-aminohexyl phosphate, 1,2-aminododecyl phosphate, hydroxypropyl phosphate, 1,4-butanediol phosphate, 3′-phosphoramidate, 5′-phosphoramidate, hexylphosphate, aminohexyl phosphate, 3′-phosphate, 5′-amino, 3′-phosphorothioate, 5′-phosphorothioate, phosphorodithioate, and bridging or non-bridging methylphosphonate or 5′-mercapto moieties (see, e.g., U.S. Pat. No. 5,998,203; Beaucage et al., Tetrahedron, 49:1925 (1993)). Non-limiting examples of phosphate backbone modifications (i.e., resulting in modified internucleotide linkages) include phosphorothioate, phosphorodithioate, methylphosphonate, phosphotriester, morpholino, amidate, carbamate, carboxymethyl, acetamidate, polyamide, sulfonate, sulfonamide, sulfamate, formacetal, thioformacetal, and alkylsilyl substitutions (see, e.g., Hunziker et al. 1995; Mesmaeker et al. 1994). Such chemical modifications can occur at the 5′-end and/or 3′-end of the sense strand, antisense strand, or both strands of the siRNA.

In other embodiments, chemical modification of the interfering RNA comprises attaching a conjugate to the chemically-modified interfering RNA molecule. The conjugate can be attached at the 5′ and/or 3′-end of the sense and/or antisense strand of the chemically-modified interfering RNA via a covalent attachment such as, e.g., a biodegradable linker. The conjugate can also be attached to the chemically-modified interfering RNA, e.g., through a carbamate group or other linking group (see, e.g., U.S. Patent Publication Nos. 20050074771, 20050043219, and 20050158727). In certain instances, the conjugate is a molecule that facilitates the delivery of the chemically-modified interfering RNA into a cell. Examples of conjugate molecules suitable for attachment to the chemically-modified interfering RNA of the present invention include, without limitation, steroids such as cholesterol, glycols such as polyethylene glycol (PEG), human serum albumin (HSA), fatty acids, carotenoids, terpenes, bile acids, folates (e.g., folic acid, folate analogs and derivatives thereof), sugars (e.g., galactose, galactosamine, N-acetyl galactosamine, glucose, mannose, fructose, fucose, etc.), phospholipids, peptides, ligands for cellular receptors capable of mediating cellular uptake, and combinations thereof (see, e.g., U.S. Patent Publication Nos. 20030130186, 20040110296, and 20040249178; U.S. Pat. No. 6,753,423). Other examples include the lipophilic moiety, vitamin, polymer, peptide, protein, nucleic acid, small molecule, oligosaccharide, carbohydrate cluster, intercalator, minor groove binder, cleaving agent, and cross-linking agent conjugate molecules described in U.S. Patent Publication Nos. 20050119470 and 20050107325. Yet other examples include the 2′-O-alkyl amine, 2′-O-alkoxyalkyl amine, polyamine, C5-cationic modified pyrimidine, cationic peptide, guanidinium group, amidininium group, cationic amino acid conjugate molecules described in U.S. Patent Publication No. 20050153337. Additional examples include the hydrophobic group, membrane active compound, cell penetrating compound, cell targeting signal, interaction modifier, and steric stabilizer conjugate molecules described in U.S. Patent Publication No. 20040167090. Further examples include the conjugate molecules described in U.S. Patent Publication No. 20050239739. The type of conjugate used and the extent of conjugation to the chemically-modified interfering RNA molecule can be evaluated for improved pharmacokinetic profiles, bioavailability, and/or stability of the interfering RNA while retaining full RNAi activity. As such, one skilled in the art can screen chemically-modified interfering RNA molecules having various conjugates attached thereto to identify ones having improved properties and full RNAi activity using any of a variety of well-known in vitro cell culture or in vivo animal models.

C. Methods

Also disclosed herein are methods of blocking the binding of an off-target molecule to an siRNA molecule, comprising, modifying at least one guanosine base of the siRNA molecule, and administering to a subject the siRNA molecule.

Also disclosed herein are methods of blocking the binding of an off-target molecule to an siRNA molecule, comprising, modifying at least two guanosine base of the siRNA molecule, and administering to a subject the siRNA molecule. For example, disclosed herein are methods of blocking the binding of an off-target molecule to an siRNA molecule, comprising, modifying at least one guanosine base of the siRNA molecule, wherein the siRNA molecule comprises two or more modified guanosine bases, and administering to a subject the siRNA molecule.

Also disclosed herein are methods of blocking the binding of an off-target molecule to an siRNA molecule, comprising, modifying at least three guanosine base of the siRNA molecule, and administering to a subject the siRNA molecule. For example, disclosed herein are methods of blocking the binding of an off-target molecule to an siRNA molecule, comprising, modifying at least one guanosine base of the siRNA molecule, wherein the siRNA molecule comprises three or more modified guanosine bases, and administering to a subject the siRNA molecule.

Also disclosed herein are methods of blocking the binding of an off-target molecule to an siRNA molecule, comprising, modifying at least one guanosine base of the siRNA molecule, and administering to a subject the siRNA molecule, wherein the modified guanosine base comprises Formula I:

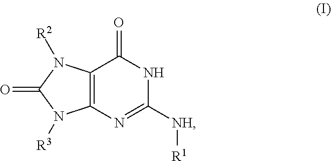

wherein R1 can be: (i) substituted or unsubstituted C1-C6 linear, branched, or cyclic alkyl; (ii) substituted or unsubstituted C2-C6 linear, branched, or cyclic alkenyl; (iii) substituted or unsubstituted C2-C6 linear or branched alkynyl; (iv) substituted or unsubstituted C6-C10 aryl; (v) substituted or unsubstituted C1-C9 heteroaryl; or (vi) substituted or unsubstituted C1-C9 heterocyclic; provided that R1 does not comprise pyrenyl, 1-oxopropyl, or tetrahydrofuranyl; wherein R2 can be: (i) substituted or unsubstituted C1-C6 linear, branched, or cyclic alkyl; (ii) substituted or unsubstituted C2-C6 linear, branched, or cyclic alkenyl; (iii) substituted or unsubstituted C2-C6 linear or branched alkynyl; (iv) substituted or unsubstituted C6-C10 aryl; (v) substituted or unsubstituted C1-C9 heteroaryl; (vi) substituted or unsubstituted C1-C9 heterocyclic; or (vii) hydrogen; wherein R3 can be: (i) substituted or unsubstituted C1-C6 linear, branched, or cyclic alkyl; (ii) substituted or unsubstituted C2-C6 linear, branched, or cyclic alkenyl; (iii) substituted or unsubstituted C2-C6 linear or branched alkynyl; (iv) substituted or unsubstituted C6-C10 aryl; (v) substituted or unsubstituted C₁-C₉ heteroaryl; (vi) substituted or unsubstituted C₁-C₉ heterocyclic; or (vii) hydrogen.

Also disclosed herein are methods of blocking the binding of an off-target molecule to an siRNA molecule, comprising, modifying at least one guanosine base of the siRNA molecule, and administering to a subject the siRNA molecule, wherein the off-target molecule is a double stranded RNA-binding motif (DSRBM).

In addition, certain siRNAs have been shown to activate the innate immune response in mammalian cells in a sequence-specific manner and are believed to occur via Toll-like receptor 7 (TLR7) present in the endosomal membrane. It appears that molecular recognition by TLR7 occurs by TLR7 making contact to the bases similar to other RNA binding proteins (Elliott et al. 1999) (FIG. 9). Disclosed herein are compositions and methods comprising modified bases that inhibit binding to TLR7, TLR8, TLR9, and related immunostimulatory proteins. For example, disclosed herein are compositions and methods comprising modified bases that inhibit binding to TLR7 and related immunostimulatory proteins, wherein the guanine base of an siRNA molecule has been altered with the introduction of N²-alkyl groups.

Also disclosed herein are methods of blocking the binding of an off-target molecule to an interfering RNA molecule, comprising, modifying at least one guanosine base of the interfering RNA molecule, and administering to a subject the interfering RNA molecule, wherein the off-target molecule is a double stranded RNA-binding motif (DSRBM), wherein the DSRBM is RNA dependent protein kinase (PKR), adenosine deaminase (ADAR), or the Toll-Like Receptor-7.

The interfering RNA described herein can be used to downregulate or silence the translation (i.e., expression) of a gene of interest. Genes of interest include, but are not limited to, genes associated with viral infection and survival, genes associated with metabolic diseases and disorders (e.g., liver diseases and disorders), genes associated with tumorigenesis and cell transformation, angiogenic genes, immunomodulator genes such as those associated with inflammatory and autoimmune responses, ligand receptor genes, and genes associated with neurodegenerative disorders.

The present invention illustrates that selective incorporation of modified guanosines into either strand of the interfering RNA duplex can reduce or completely abrogate the off-target response to synthetic interfering RNA. Modified interfering RNA targeting can mediate potent silencing of its target mRNA. Advantageously, the approach to interfering RNA design and delivery described herein is widely applicable and advances synthetic interfering RNA into a broad range of therapeutic areas. For example, disclosed herein is a method of synthesizing 2′-deoxy-N²-alkyl-7,8-dihydro-8-oxoguanosines as cyanoethylphosphoramidites wherein “alkyl” is n-propyl or benzyl or, for the purposes of comparison, hydrogen and wherein many other alkyl groups can be envisioned by the same synthetic route. The modified guanosines, X, are individually incorporated into synthetic RNA oligonucleotides at one or more positions in which a single X:C base pair replaces a U:A base pair in the antisense:sense duplex.

Accordingly, in an aspect, the present invention relates to a pharmaceutical composition comprising a modified interfering RNA according to the disclosed methods and compositions and a pharmaceutically acceptable diluent, carrier or adjuvant. In another aspect, the present invention relates to a modified interfering RNA as disclosed herein for use as a medicament.

As will be understood dosing is dependent on severity and responsiveness of the disease state to be treated, and the course of treatment lasting from several days to several months, or until a cure is effected or a diminution of the disease state is achieved. Optimal dosing schedules can be calculated from measurements of drug accumulation in the body of the patient. Optimum dosages may vary depending on the relative potency of individual interfering RNAs. Generally it can be estimated based on EC50s found to be effective in in vitro and in vivo animal models. In general, dosage is from 0.01 μg to 1 g per kg of body weight, and may be given once or more daily, weekly, monthly or yearly, or even once every 2 to 10 years or by continuous infusion for hours up to several months. The repetition rates for dosing can be estimated based on measured residence times and concentrations of the drug in bodily fluids or tissues. Following successful treatment, it may be desirable to have the patient undergo maintenance therapy to prevent the recurrence of the disease state.

As indicated above the invention also relates to a pharmaceutical composition, which comprises at least one modified interfering RNA of the invention as an active ingredient. It should be understood that the pharmaceutical composition according to the invention optionally comprises a pharmaceutical carrier, and that the pharmaceutical composition optionally comprises further compounds, such as chemotherapeutic compounds, anti-inflammatory compounds, antiviral compounds and/or immuno-modulating compounds.

The modified interfering RNAs of the invention can be used “as is” or in form of a variety of pharmaceutically acceptable salts. As used herein, the term “pharmaceutically acceptable salts” refers to salts that retain the desired biological activity of the herein-identified modified interfering RNAs and exhibit minimal undesired toxicological effects. Non-limiting examples of such salts can be formed with organic amino acid and base addition salts formed with metal cations such as zinc, calcium, bismuth, barium, magnesium, aluminum, copper, cobalt, nickel, cadmium, sodium, potassium, and the like, or with a cation formed from ammonia, N,N-dibenzylethylene-diamine, D-glucosamine, tetraethylammonium, or ethylenediamine.

In one embodiment of the invention the modified interfering RNA may be in the form of a pro-drug. Oligonucleotides are by virtue negatively charged ions. Due to the lipophilic nature of cell membranes the cellular uptake of oligonucleotides are reduced compared to neutral or lipophilic equivalents. This polarity “hindrance” can be avoided by using the pro-drug approach (see, e.g., Crooke 1998). In this approach the oligonucleotides are prepared in a protected manner so that the oligo is neutral when it is administered. These protection groups are designed in such a way that they can be removed when the oligo is taken up by the cells. Examples of such protection groups are S-acetylthioethyl (SATE) or S-pivaloylthioethyl (t-butyl-SATE). These protection groups are nuclease resistant and are selectively removed intracellulary.

Pharmaceutically acceptable binding agents and adjuvants may comprise part of the formulated drug. Capsules, tablets and pills etc. may contain for example the following compounds: microcrystalline cellulose, gum or gelatin as binders; starch or lactose as excipients; stearates as lubricants; various sweetening or flavouring agents. For capsules the dosage unit may contain a liquid carrier like fatty oils. Likewise coatings of sugar or enteric agents may be part of the dosage unit. The oligonucleotide formulations may also be emulsions of the active pharmaceutical ingredients and a lipid forming a micellular emulsion. A compound of the invention may be mixed with any material that do not impair the desired action, or with material that supplement the desired action. These could include other drugs including other nucleotide compounds. For parenteral, subcutaneous, intradermal or topical administration the formulation may include a sterile diluent, buffers, regulators of tonicity and antibacterials. The active compound may be prepared with carriers that protect against degradation or immediate elimination from the body, including implants or microcapsules with controlled release properties. For intravenous administration the preferred carriers are physiological saline or phosphate buffered saline. Preferably, an oligomeric compound is included in a unit formulation such as in a pharmaceutically acceptable carrier or diluent in an amount sufficient to deliver to a patient a therapeutically effective amount without causing serious side effects in the treated patient.

The pharmaceutical compositions of the present invention may be administered in a number of ways depending upon whether local or systemic treatment is desired and upon the area to be treated. Administration may be (a) oral (b) pulmonary, e.g., by inhalation or insufflation of powders or aerosols, including by nebulizer; intratracheal, intranasal, (c) topical including epidermal, transdermal, ophthalmic and to mucous membranes including vaginal and rectal delivery; or (d) parenteral including intravenous, intraarterial, subcutaneous, intraperitoneal or intramuscular injection or infusion; or intracranial, e.g., intrathecal or intraventricular, administration. In one embodiment the pharmaceutical composition is administered IV, IP, orally, topically or as a bolus injection or administered directly in to the target organ. Pharmaceutical compositions and formulations for topical administration may include transdermal patches, ointments, lotions, creams, gels, drops, sprays, suppositories, liquids and powders. Conventional pharmaceutical carriers, aqueous, powder or oily bases, thickeners and the like may be necessary or desirable. Coated condoms, gloves and the like may also be useful. Preferred topical formulations include those in which the compounds of the invention are in admixture with a topical delivery agent such as lipids, liposomes, fatty acids, fatty acid esters, steroids, chelating agents and surfactants. Compositions and formulations for oral administration include but is not restricted to powders or granules, microparticulates, nanoparticulates, suspensions or solutions in water or non-aqueous media, capsules, gel capsules, sachets, tablets or minitablets. Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids. Delivery of drug to tumour tissue may be enhanced by carrier-mediated delivery including, but not limited to, cationic liposomes, cyclodextrins, porphyrin derivatives, branched chain dendrimers, polyethylenimine polymers, nanoparticles and microspheres (Dass 2002). The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product. The compositions of the present invention may be formulated into any of many possible dosage forms such as, but not limited to, tablets, capsules, gel capsules, liquid syrups, soft gels and suppositories. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The compounds of the invention may also be conjugated to active drug substances, for example, aspirin, ibuprofen, a sulfa drug, an antidiabetic, an antibacterial or an antibiotic.

The compounds disclosed herein are useful for a number of therapeutic applications as indicated above. In general, therapeutic methods of the invention include administration of a therapeutically effective amount of a modified interfering RNA to a mammal, particularly a human. In a certain embodiment, the present invention provides pharmaceutical compositions containing (a) one or more compounds of the invention, and (b) one or more chemotherapeutic agents. When used with the compounds of the invention, such chemotherapeutic agents may be used individually, sequentially, or in combination with one or more other such chemotherapeutic agents or in combination with radiotherapy. All chemotherapeutic agents known to a person skilled in the art are here incorporated as combination treatments with compound according to the invention. Other active agents, such as anti-inflammatory drugs, including but not limited to nonsteroidal anti-inflammatory drugs and corticosteroids, antiviral drugs, and immuno-modulating drugs may also be combined in compositions of the invention. Two or more combined compounds may be used together or sequentially.

Also disclosed are methods for using a modified interfering RNA according to the invention for the manufacture of a medicament for the treatment of cancer. In another aspect the present invention concerns a method for treatment of, or prophylaxis against, cancer, said method comprising administering a modified interfering RNA of the invention or a pharmaceutical composition of the invention to a patient in need thereof.

Such cancers may include lymphoreticular neoplasia, lymphoblastic leukemia, brain tumors, gastric tumors, plasmacytomas, multiple myeloma, leukemia, connective tissue tumors, lymphomas, and solid tumors.

In the use of a compound of the invention for the manufacture of a medicament for the treatment of cancer, said cancer may suitably be in the form of a solid tumor. Analogously, in the method for treating cancer disclosed herein said cancer may suitably be in the form of a solid tumor.

Furthermore, said cancer is also suitably a carcinoma. The carcinoma is typically selected from the group consisting of malignant melanoma, basal cell carcinoma, ovarian carcinoma, breast carcinoma, non-small cell lung cancer, renal cell carcinoma, bladder carcinoma, recurrent superficial bladder cancer, stomach carcinoma, prostatic carcinoma, pancreatic carcinoma, lung carcinoma, cervical carcinoma, cervical dysplasia, laryngeal papillomatosis, colon carcinoma, colorectal carcinoma and carcinoid tumors. More typically, said carcinoma is selected from the group consisting of malignant melanoma, non-small cell lung cancer, breast carcinoma, colon carcinoma and renal cell carcinoma. The malignant melanoma is typically selected from the group consisting of superficial spreading melanoma, nodular melanoma, lentigo maligna melanoma, acral melagnoma, amelanotic melanoma and desmoplastic melanoma.

Alternatively, the cancer may suitably be a sarcoma. The sarcoma is typically in the form selected from the group consisting of osteosarcoma, Ewing's sarcoma, chondrosarcoma, malignant fibrous histiocytoma, fibrosarcoma and Kaposi's sarcoma. Alternatively, the cancer may suitably be a glioma.

Also disclosed is a method of using a modified interfering RNA according to the invention for the manufacture of a medicament for the treatment of cancer, wherein said medicament further comprises a chemotherapeutic agent selected from the group consisting of adrenocorticosteroids, such as prednisone, dexamethasone or decadron; altretamine (hexalen, hexamethylmelamine (HMM)); amifostine (ethyol); aminoglutethimide (cytadren); amsacrine (M-AMSA); anastrozole (arimidex); androgens, such as testosterone; asparaginase (elspar); bacillus calmette-gurin; bicalutamide (casodex); bleomycin (blenoxane); busulfan (myleran); carboplatin (paraplatin); carmustine (BCNU, BiCNU); chlorambucil (leukeran); chlorodeoxyadenosine (2-CDA, cladribine, leustatin); cisplatin (platinol); cytosine arabinoside (cytarabine); dacarbazine (DTIC); dactinomycin (actinomycin-D, cosmegen); daunorubicin (cerubidine); docetaxel (taxotere); doxorubicin (adriomycin); epirubicin; estramustine (emcyt); estrogens, such as diethylstilbestrol (DES); etopside (VP-16, VePesid, etopophos); fludarabine (fludara); flutamide (eulexin); 5-FUDR (floxuridine); 5-fluorouracil (5-FU); gemcitabine (gemzar); goserelin (zodalex); herceptin (trastuzumab); hydroxyurea (hydrea); idarubicin (idamycin); ifosfamide; IL-2 (proleukin, aldesleukin); interferon alpha (intron A, roferon A); irinotecan (camptosar); leuprolide (lupron); levamisole (ergamisole); lomustine (CCNU); mechlorathamine (mustargen, nitrogen mustard); melphalan (alkeran); mercaptopurine (purinethol, 6-MP); methotrexate (mexate); mitomycin-C (mutamucin); mitoxantrone (novantrone); octreotide (sandostatin); pentostatin (2-deoxycoformycin, nipent); plicamycin (mithramycin, mithracin); prorocarbazine (matulane); streptozocin; tamoxifin (nolvadex); taxol (paclitaxel); teniposide (vumon, VM-26); thiotepa; topotecan (hycamtin); tretinoin (vesanoid, all-trans retinoic acid); vinblastine (valban); vincristine (oncovin) and vinorelbine (navelbine). Suitably, the further chemotherapeutic agent is selected from taxanes such as Taxol, Paclitaxel or Docetaxel.

Similarly, the invention is further directed to the use of a modified interfering RNA according to the invention for the manufacture of a medicament for the treatment of cancer, wherein said treatment further comprises the administration of a further chemotherapeutic agent selected from the group consisting of adrenocorticosteroids, such as prednisone, dexamethasone or decadron; altretamine (hexalen, hexamethylmelamine (HMM)); amifostine (ethyol); aminoglutethimide (cytadren); amsacrine (M-AMSA); anastrozole (arimidex); androgens, such as testosterone; asparaginase (elspar); bacillus calmette-gurin; bicalutamide (casodex); bleomycin (blenoxane); busulfan (myleran); carboplatin (paraplatin); carmustine (BCNU, BiCNU); chlorambucil (leukeran); chlorodeoxyadenosine (2-CDA, cladribine, leustatin); cisplatin (platinol); cytosine arabinoside (cytarabine); dacarbazine (DTIC); dactinomycin (actinomycin-D, cosmegen); daunorubicin (cerubidine); docetaxel (taxotere); doxorubicin (adriomycin); epirubicin; estramustine (emcyt); estrogens, such as diethylstilbestrol (DES); etopside (VP-16, VePesid, etopophos); fludarabine (fludara); flutamide (eulexin); 5-FUDR (floxuridine); 5-fluorouracil (5-FU); gemcitabine (gemzar); goserelin (zodalex); herceptin (trastuzumab); hydroxyurea (hydrea); idarubicin (idamycin); ifosfamide; IL-2 (proleukin, aldesleukin); interferon alpha (intron A, roferon A); irinotecan (camptosar); leuprolide (lupron); levamisole (ergamisole); lomustine (CCNU); mechlorathamine (mustargen, nitrogen mustard); melphalan (alkeran); mercaptopurine (purinethol, 6-MP); methotrexate (mexate); mitomycin-C (mutamucin); mitoxantrone (novantrone); octreotide (sandostatin); pentostatin (2-deoxycoformycin, nipent); plicamycin (mithramycin, mithracin); prorocarbazine (matulane); streptozocin; tamoxifin (nolvadex); taxol (paclitaxel); teniposide (vumon, VM-26); thiotepa; topotecan (hycamtin); tretinoin (vesanoid, all-trans retinoic acid); vinblastine (valban); vincristine (oncovin) and vinorelbine (navelbine). Suitably, said treatment further comprises the administration of a further chemotherapeutic agent selected from taxanes, such as Taxol, Paclitaxel or Docetaxel.

Alternatively stated, the invention is furthermore directed to a method for treating cancer, said method comprising administering a modified interfering RNA of the invention or a pharmaceutical composition according to the invention to a patient in need thereof and further comprising the administration of a further chemotherapeutic agent. Said further administration may be such that the further chemotherapeutic agent is conjugated to the compound of the invention, is present in the pharmaceutical composition, or is administered in a separate formulation.

In a particular interesting embodiment of the invention, the modified interfering RNA compounds according to the invention are used for targeting Severe Acute Respiratory Syndrome (SARS), which first appeared in China in November 2002. According to the WHO over 8,000 people have been infected world-wide, resulting in over 900 deaths. A previously unknown coronavirus has been identified as the causative agent for the SARS epidemic (Drosten et al. 2003; Fouchier et al. 2003). Identification of the SARS-COV was followed by rapid sequencing of the viral genome of multiple isolates (Ruan et al. 2003; Rota et al. 2003; Marra 2003). This sequence information immediately made possible the development of SARS antivirals by nucleic acid-based knock-down techniques such as interfering RNA. The nucleotide sequence encoding the SARS-COV RNA-dependent RNA polymerase (Pol) is highly conserved throughout the coronavirus family. The Pol gene product is translated from the genomic RNA as a part of a polyprotein, and uses the genomic RNA as a template to synthesize negative-stranded RNA and subsequently sub-genomic mRNA. The Pol protein is thus expressed early in the viral life cycle and is crucial to viral replication.

Accordingly, in a further another aspect the present invention relates the use of a modified interfering RNA according to the invention for the manufacture of a medicament for the treatment of Severe Acute Respiratory Syndrome (SARS), as well as to a method for treating Severe Acute Respiratory Syndrome (SARS), said method comprising administering a modified interfering RNA according to the invention or a pharmaceutical composition according to the invention to a patient in need thereof.

It is contemplated that the compounds of the invention may be broadly applicable to a broad range of infectious diseases, such as diphtheria, tetanus, pertussis, polio, hepatitis B, hemophilus influenza, measles, mumps, and rubella.

Accordingly, in yet another aspect the present invention relates the use of a modified interfering RNA according to the invention for the manufacture of a medicament for the treatment of an infectious disease, as well as to a method for treating an infectious disease, said method comprising administering a modified interfering RNA according to the invention or a pharmaceutical composition according to the invention to a patient in need thereof.

The inflammatory response is an essential mechanism of defense of the organism against the attack of infectious agents, and it is also implicated in the pathogenesis of many acute and chronic diseases, including autoimmune disorders. In spite of being needed to fight pathogens, the effects of an inflammatory burst can be devastating. It is therefore often necessary to restrict the symptomatology of inflammation with the use of anti-inflammatory drugs. Inflammation is a complex process normally triggered by tissue injury that includes activation of a large array of enzymes, the increase in vascular permeability and extravasation of blood fluids, cell migration and release of chemical mediators, all aimed to both destroy and repair the injured tissue.

In yet another aspect, the present invention relates to the use of a modified interfering RNA according to the invention for the manufacture of a medicament for the treatment of an inflammatory disease, as well as to a method for treating an inflammatory disease, said method comprising administering a modified interfering RNA according to the invention or a pharmaceutical composition according to the invention to a patient in need thereof.

The inflammatory disease can be a rheumatic disease and/or a connective tissue diseases, such as rheumatoid arthritis, systemic lupus erythematous (SLE) or Lupus, scleroderma, polymyositis, inflammatory bowel disease, dermatomyositis, ulcerative colitis, Crohn's disease, vasculitis, psoriatic arthritis, exfoliative psoriatic dermatitis, pemphigus vulgaris, Sjorgren's syndrome, inflammatory bowel disease, and Crohn's disease.

The inflammatory disease can also be a non-rheumatic inflammation, like bursitis, synovitis, capsulitis, tendinitis and/or other inflammatory lesions of traumatic and/or sportive origin.

The modified interfering RNAs of the present invention can be utilized for as research reagents for diagnostics, therapeutics and prophylaxis. In research, the modified interfering RNA can be used to specifically inhibit the synthesis of target genes in cells and experimental animals thereby facilitating functional analysis of the target or an appraisal of its usefulness as a target for therapeutic intervention. In diagnostics, the modified interfering RNA can be used to detect and quantitate target expression in cell and tissues by Northern blotting, in-situ hybridisation or similar techniques. For therapeutics, an animal or a human, suspected of having a disease or disorder, which modulating the expression of target can treat is treated by administering the modified interfering RNA compounds in accordance with this invention. Further provided are methods of treating an animal particular mouse and rat and treating a human, suspected of having or being prone to a disease or condition, associated with expression of target by administering a therapeutically or prophylactically effective amount of one or more of the modified interfering RNA compounds or compositions of the invention.

D. Kits

The compositions and materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for reducing or completely abrogating the off-target response to synthetic interfering RNA, the kit comprising one or more reagent compositions and one or more components or reagents for capture of the target nucleic acid, tHDA amplification, detection of amplification products, or both. For example, the kits can include one or more compounds of Formula I:

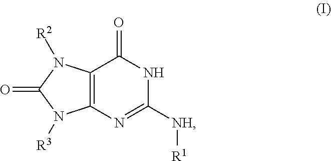

wherein R1 can be: (i) substituted or unsubstituted C1-C6 linear, branched, or cyclic alkyl; (ii) substituted or unsubstituted C2-C6 linear, branched, or cyclic alkenyl; (iii) substituted or unsubstituted C2-C6 linear or branched alkynyl; (iv) substituted or unsubstituted C6-C10 aryl; (v) substituted or unsubstituted C1-C9 heteroaryl; or (vi) substituted or unsubstituted C1-C9 heterocyclic; provided that R1 does not comprise pyrenyl, 1-oxopropyl, or tetrahydrofuranyl; wherein R2 can be: (i) substituted or unsubstituted C1-C6 linear, branched, or cyclic alkyl; (ii) substituted or unsubstituted C2-C6 linear, branched, or cyclic alkenyl; (iii) substituted or unsubstituted C2-C6 linear or branched alkynyl; (iv) substituted or unsubstituted C6-C10 aryl; (v) substituted or unsubstituted C1-C9 heteroaryl; (vi) substituted or unsubstituted C1-C9 heterocyclic; or (vii) hydrogen; wherein R3 can be: (i) substituted or unsubstituted C1-C6 linear, branched, or cyclic alkyl; (ii) substituted or unsubstituted C2-C6 linear, branched, or cyclic alkenyl; (iii) substituted or unsubstituted C2-C6 linear or branched alkynyl; (iv) substituted or unsubstituted C6-C10 aryl; (v) substituted or unsubstituted C1-C9 heteroaryl; (vi) substituted or unsubstituted C1-C9 heterocyclic; or (vii) hydrogen.

Also disclosed herein kits comprising compounds of Formula I: wherein R³ is a residue of Formula II:

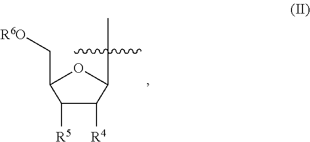

wherein R⁴ can be: (i) hydrogen; (ii) hydroxyl; (iii) alkoxy; (iv) amino; or (v) halogen; wherein R⁵ can be: (i) hydrogen; (ii) hydroxyl; (iii) alkoxy; (iv) amino; (v) halogen; (vi) C₁-C₁₂ phosphonite, phosphate, phosphonate, or phosphoryl; or (vii) an O-linked solid support; and wherein R⁶ is: (i) hydrogen; (ii) a protecting group; (iii) a monophosphate; (iv) a diphosphate; (v) a triphosphate; (vi) a nucleotide; or (vii) a deoxynucleotide.

Also disclosed herein kits comprising nucleosides of Formula III:

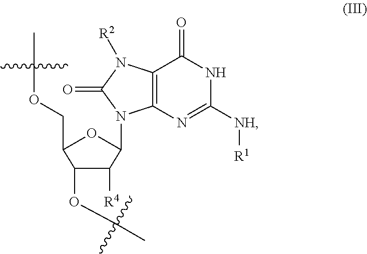

wherein R¹ can be: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; (v) substituted or unsubstituted C₁-C₉ heteroaryl; or (vi) substituted or unsubstituted C₁-C₉ heterocyclic; provided that R¹ does not comprise pyrenyl, 1-oxopropyl, or tetrahydrofuranyl; wherein R² can be: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; (v) substituted or unsubstituted C₁-C₉ heteroaryl; (vi) substituted or unsubstituted C₁-C₉ heterocyclic; or (vii) hydrogen; and wherein R⁴ can be: (i) hydrogen; (ii) hydroxyl; (iii) alkoxy; (vi) amino; or (v) halogen.

Also disclosed herein kits comprising an oligonucleotide comprising at least one of Formula VI:

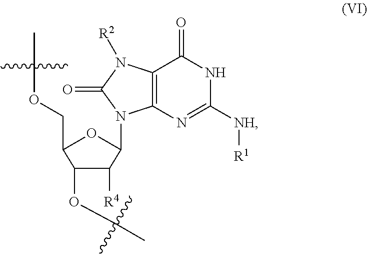

wherein R¹ can be: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; (v) substituted or unsubstituted C₁-C₉ heteroaryl; or (vi) substituted or unsubstituted C₁-C₉ heterocyclic; provided that R¹ does not comprise pyrenyl, 1-oxopropyl, or tetrahydrofuranyl; wherein R² can be: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; (v) substituted or unsubstituted C₁-C₉ heteroaryl; (vi) substituted or unsubstituted C₁-C₉ heterocyclic; or (vii) hydrogen; and wherein R⁴ can be: (i) hydrogen; (ii) hydroxyl; (iii) alkoxy; (iv) amino; or (v) halogen.

Also disclosed herein kits comprising an polynucleotide comprising at least one of Formula VI:

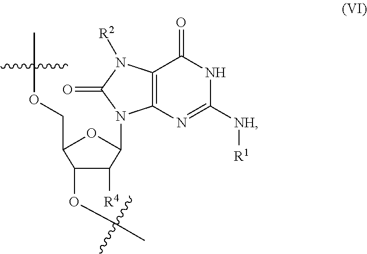

wherein R¹ can be: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; (v) substituted or unsubstituted C₁-C₉ heteroaryl; or (vi) substituted or unsubstituted C₁-C₉ heterocyclic; provided that R¹ does not comprise pyrenyl, 1-oxopropyl, or tetrahydrofuranyl; wherein R² can be: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; (v) substituted or unsubstituted C₁-C₉ heteroaryl; (vi) substituted or unsubstituted C₁-C₉ heterocyclic; or (vii) hydrogen; and wherein R⁴ can be: (i) hydrogen; (ii) hydroxyl; (iii) alkoxy; (iv) amino; or (v) halogen.

Also disclosed herein are sets of nucleotides comprising compounds of Formula I: wherein R³ is a residue of Formula II:

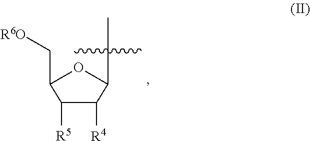

wherein R⁴ can be: (i) hydrogen; (ii) hydroxyl; (iii) alkoxy; (iv) amino; or (v) halogen; wherein R⁵ can be: (i) hydrogen; (ii) hydroxyl; (iii) alkoxy; (iv) amino; (v) halogen; (vi) C₁-C₁₂ phosphonite, phosphate, phosphonate, or phosphoryl; or (vii) an O-linked solid support; and wherein R⁶ is: (i) dimethoxytrityl (DMT); (ii) monomethoxytrityl; (iii) 9-phenylxanthen-9-yl (Pixyl); or (iv) 9-(p-methoxyphenyl)xanthen-9-yl (Mox).

Also disclosed herein are sets of nucleotides comprising at least one oligonucleotide or polynucleotide comprising Formula VI:

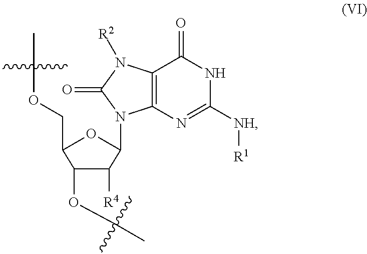

wherein R¹ can be: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; (v) substituted or unsubstituted C₁-C₉ heteroaryl; or (vi) substituted or unsubstituted C₁-C₉ heterocyclic; provided that R¹ does not comprise pyrenyl, 1-oxopropyl, or tetrahydrofuranyl; wherein R² can be: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; (v) substituted or unsubstituted C₁-C₉ heteroaryl; (vi) substituted or unsubstituted C₁-C₉ heterocyclic; or (vii) hydrogen; and wherein R⁴ can be: (i) hydrogen; (ii) hydroxyl; (iii) alkoxy; (iv) amino; or (v) halogen.

E. Mixtures

Disclosed are mixtures formed by preparing the disclosed composition or performing or preparing to perform the disclosed methods. Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

F. Systems

Disclosed are systems useful for performing, or aiding in the performance of, the disclosed methods. Also disclosed are systems for producing reagent compositions. Systems generally comprise combinations of articles of manufacture such as structures, machines, devices, and the like, and compositions, compounds, materials, and the like. Such combinations that are disclosed or that are apparent from the disclosure are contemplated. For example, disclosed and contemplated are systems comprising solid supports and reagent compositions.

G. Data Structures and Computer Control

Disclosed are data structures used in, generated by, or generated from, the disclosed method. Data structures generally are any form of data, information, and/or objects collected, organized, stored, and/or embodied in a composition or medium. A target fingerprint stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

Disclosed herein is a nucleobase represented by the formula:

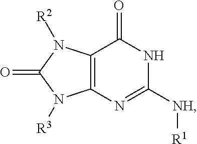

wherein R¹ is: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; v) substituted or unsubstituted C₁-C₉ heteroaryl; or (vi) substituted or unsubstituted C₁-C₉ heterocyclic; provided that R¹ does not comprise pyrenyl, 1-oxopropyl, or tetrahydrofuranyl; each R² and R³ is independently: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; (v) substituted or unsubstituted C₁-C₉ heteroaryl; (vi) substituted or unsubstituted C₁-C₉ heterocyclic; or (vii) hydrogen. R¹ can be substituted or unsubstituted methyl, ethyl, n-propyl, iso-propyl, n-butyl, iso-butyl, sec-butyl, tent-butyl, or benzyl. R² can be hydrogen. R³ can be substituted or unsubstituted tetrahydrofuranyl or tetrahydropyranyl. R³ can be represented by the formula:

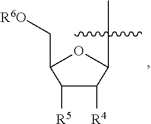

wherein R⁴ is: (i) hydrogen; (ii) hydroxyl; (iii) alkoxy; iv) amino; or (v) halogen; R⁵ is: (i) hydrogen; (ii) hydroxyl; (iii) alkoxy; (iv) amino; (v) halogen; (vi) C₁-C₁₂phosphonite, phosphate, phosphonate, or phosphoryl; or (vii) an O-linked solid support; and R⁶ is: (i) hydrogen; (ii) a protecting group; or (iii) a nucleoside; or (iv) a deoxynucleoside. R⁵ can be (i) —O—(N,N-diisopropyl O-methyl phosphoramidite); or —O—(N,N-diisopropyl —O-2-cyanoethyl phosphoramidite). R⁶ can be (i) dimethoxytrityl (DMT); (ii) monomethoxytrityl; (iii) 9-phenylxanthen-9-yl (Pixyl); or (iv) 9-(p-methoxyphenyl)xanthen-9-yl (Mox).

Also disclosed is a method for making an alkylated nucleobase, comprising: a) providing a compound represented by the formula:

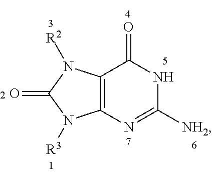

wherein each R² and R³ is independently: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; (v) substituted or unsubstituted C₁-C₉ heteroaryl; (vi) substituted or unsubstituted C₁-C₉ heterocyclic; or (vii) hydrogen; and b) alkylating the amino group at the 6 position of the compound of step a to provide an alkylated nucleobase represented by the formula:

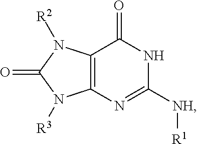

wherein R¹ is: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; (v) substituted or unsubstituted C₁-C₉ heteroaryl; or (vi) substituted or unsubstituted C₁-C₉ heterocyclic; provided that R¹ does not comprise pyrenyl, 1-oxopropyl, or tetrahydrofuranyl.

Alkylating the amino group at the 6 position of the compound of step a can comprise c) reacting the compound of step a with a compound represented by the formula R¹CHO, wherein R¹ is: (i) substituted or unsubstituted C₁-C₆ linear, branched, or cyclic alkyl; (ii) substituted or unsubstituted C₂-C₆ linear, branched, or cyclic alkenyl; (iii) substituted or unsubstituted C₂-C₆ linear or branched alkynyl; (iv) substituted or unsubstituted C₆-C₁₀ aryl; (v) substituted or unsubstituted C₁-C₉ heteroaryl; or (vi) substituted or unsubstituted C₁-C₉ heterocyclic; provided that R¹ does not comprise pyrenyl, 1-oxopropyl, or tetrahydrofuranyl; and d) reducing the product of step b to provide the alkylated nucleobase.

Alkylating the amino group at the 6 position of the compound of step a) can comprise reacting the compound of step a with a compound represented by the formula R¹X, wherein R¹ is: i) substituted or unsubstituted C₁-C₆ linear, branched, or cyclic alkyl; ii) substituted or unsubstituted C₂-C₆ linear, branched, or cyclic alkenyl; iii) substituted or unsubstituted C₂-C₆ linear or branched alkynyl; iv) substituted or unsubstituted C₆-C₁₀ aryl; v) substituted or unsubstituted C₁-C₉ heteroaryl; or vi) substituted or unsubstituted C₁-C₉ heterocyclic; provided that R¹ does not comprise pyrenyl, 1-oxopropyl, or tetrahydrofuranyl; and X is Br, I, F, or Cl.

Also disclosed is a nucleic acid strand comprising a residue represented by the formula:

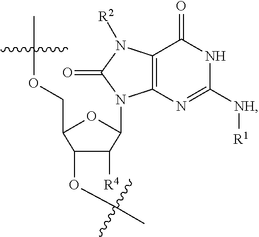

wherein R¹ is: i) substituted or unsubstituted C₁-C₆ linear, branched, or cyclic alkyl; ii) substituted or unsubstituted C₂-C₆ linear, branched, or cyclic alkenyl; iii) substituted or unsubstituted C₂-C₆ linear or branched alkynyl; iv) substituted or unsubstituted C₆-C₁₀ aryl; v) substituted or unsubstituted C₁-C₉ heteroaryl; vi) substituted or unsubstituted C₁-C₉ heterocyclic; provided that R¹ does not comprise pyrenyl, 1-oxopropyl, or tetrahydrofuranyl; R² is: i) substituted or unsubstituted C₁-C₆ linear, branched, or cyclic alkyl; ii) substituted or unsubstituted C₂-C₆ linear, branched, or cyclic alkenyl; iii) substituted or unsubstituted C₂-C₆ linear or branched alkynyl; iv) substituted or unsubstituted C₆-C₁₀ aryl; v) substituted or unsubstituted C₁-C₉ heteroaryl; vi) substituted or unsubstituted C₁-C₉ heterocyclic; or vii) hydrogen; and R⁴ is: i) hydrogen; ii) hydroxyl; iii) alkoxy; iv) amino; or v) halogen.

The nucleobases of the invention are represented by the formula:

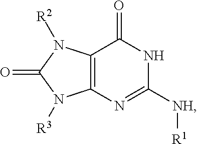

wherein R¹ is: i) substituted or unsubstituted C₁-C₆ linear, branched, or cyclic alkyl; ii) substituted or unsubstituted C₂-C₆ linear, branched, or cyclic alkenyl; iii) substituted or unsubstituted C₂-C₆ linear or branched alkynyl; iv) substituted or unsubstituted C₆-C₁₀ aryl; v) substituted or unsubstituted C₁-C₉ heteroaryl; or vi) substituted or unsubstituted C₁-C₉ heterocyclic; provided that R¹ does not comprise pyrenyl, 1-oxopropyl, or tetrahydrofuranyl; each R² and R³ is independently: i) substituted or unsubstituted C₁-C₆ linear, branched, or cyclic alkyl; ii) substituted or unsubstituted C₂-C₆ linear, branched, or cyclic alkenyl; iii) substituted or unsubstituted C₂-C₆ linear or branched alkynyl; iv) substituted or unsubstituted C₆-C₁₀ aryl; v) substituted or unsubstituted C₁-C₉ heteroaryl; vi) substituted or unsubstituted C₁-C₉ heterocyclic; or vii) hydrogen.

Generally, R¹ can comprise any suitable group that would sterically hinder the binding of the nucleobase with a cellular double-stranded RNA-binding protein. With reference to FIG. 9, for example, R¹ (labeled R in FIG. 9) of an exemplary OdG-U rich siRNA strand can effectively inhibit the binding of the OdG-U rich siRNA strand with the Toll-like receptor 7 (TLR7) immune gene, thereby avoiding an undesirable immune response in a subject that has been administered the OdG-U rich siRNA strand. In specific embodiments, R¹ is substituted or unsubstituted methyl, ethyl, n-propyl, iso-propyl, n-butyl, iso-butyl, sec-butyl, tent-butyl, or benzyl.

The substituent R² can comprise a variety of groups, depending on the desired mode of action of the nucleobase. With reference to FIG. 5, an exemplary nucleobase can bind in the minor groove of RNA with C in a typical Watson-Crick pairing. In this example, the substituent at R² is not involved in the pairing and can thus be any of those groups defined above. However, again with reference to FIG. 5, a Hoogsten pairing between the nucleobase of the invention and A involves the substituent at R² as a hydrogen bond donor. Thus, in this example, R² is preferably hydrogen.

The substituent R³ can generally comprise any suitable group, but typically comprises a cyclic group. Specific examples include without limitation substituted or unsubstituted tetrahydrofuranyl or tetrahydropyranyl. In one embodiment, R³ is represented by the formula:

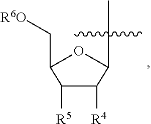

wherein R⁴ is i) hydrogen; ii) hydroxyl; iii) alkoxy; iv) amino; or v) halogen; R⁵ is: i) hydrogen; ii) hydroxyl; iii) alkoxy; iv) amino; or v) halogen; vi) C₁-C₁₂phosphonite, phosphate, phosphonate, or phosphoryl; vii) an O-linked solid support; and R⁶ is: i) hydrogen; ii) a protecting group; or iii) a nucleoside; or iv) a deoxynucleoside. In various embodiments, the nucleobase can be in oxyribose or deoxyribose form, and as such R⁴ can be hydroxyl, alkoxy, protected hydroxyl, or hydrogen.

When R5 comprises a C1-C12 phosphonite, phosphate, phosphonate, or phosphoryl group, phosphonite, phosphate, phosphonate, or phosphoryl group can be protected with a suitable protecting group. Protecting groups for such residues are attached to the phosphorus-bound oxygen, and serve to protect the phosphorus during oligonucleotide synthesis. See, for example, Oligonucleotides and Analogues: A Practical Approach, Eckstein, F., Ed., IRL Press, Oxford, U.K. 1991, which is incorporated herein by this reference, for its teachings of phosphonite, phosphate, phosphonate, and phosphoryl protecting groups. One exemplary phosphoryl protecting group is the cyanoethyl group. Other exemplary phosphoryl protecting groups include 4-cyano-2-butenyl groups, methyl groups, and diphenylmethylsilylethyl (DPSE) groups. In one specific embodiment, R5 can comprise —O—(N,N-diisopropyl O-methyl phosphoramidite) or —O—(N,N-diisopropyl O-2-cyanoethyl phosphoramidite). These two groups, for example, are suitable for use when incorporating the nucleobase into a nucleic acid strand, such as RNA.

When the nucleobase is present in a strand of a nucleic acid, R⁵ can be hydroxyl if the nucleobase terminates the strand, or R⁵ can be a suitable nucleoside. When R⁵ is hydroxyl, it can be protected. Thus, in various embodiments, a disclosed nucleic acid strand, such as a strand of RNA, can comprise a structural residue represented by the formula:

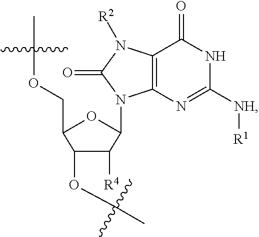

wherein R¹ is: i) substituted or unsubstituted C₁-C₆ linear, branched, or cyclic alkyl; ii) substituted or unsubstituted C₂-C₆ linear, branched, or cyclic alkenyl; iii) substituted or unsubstituted C₂-C₆ linear or branched alkynyl; iv) substituted or unsubstituted C₆-C₁₀ aryl; v) substituted or unsubstituted C₁-C₉ heteroaryl; vi) substituted or unsubstituted C₁-C₉ heterocyclic; provided that R¹ does not comprise pyrenyl, 1-oxopropyl, or tetrahydrofuranyl; R² is: i) substituted or unsubstituted C₁-C₆ linear, branched, or cyclic alkyl; ii) substituted or unsubstituted C₂-C₆ linear, branched, or cyclic alkenyl; iii) substituted or unsubstituted C₂-C₆ linear or branched alkynyl; iv) substituted or unsubstituted C₆-C₁₀ aryl; v) substituted or unsubstituted C₁-C₉ heteroaryl; vi) substituted or unsubstituted C₁-C₉ heterocyclic; or vii) hydrogen; and R⁴ is: i) hydrogen; or ii) hydroxyl.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Modified siRNA Molecules

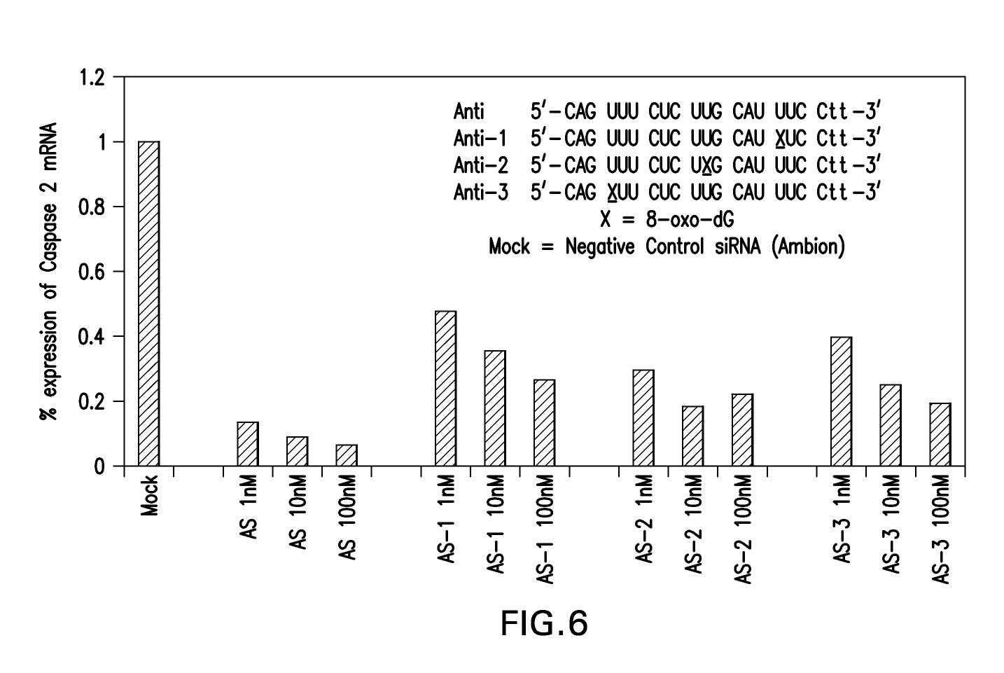

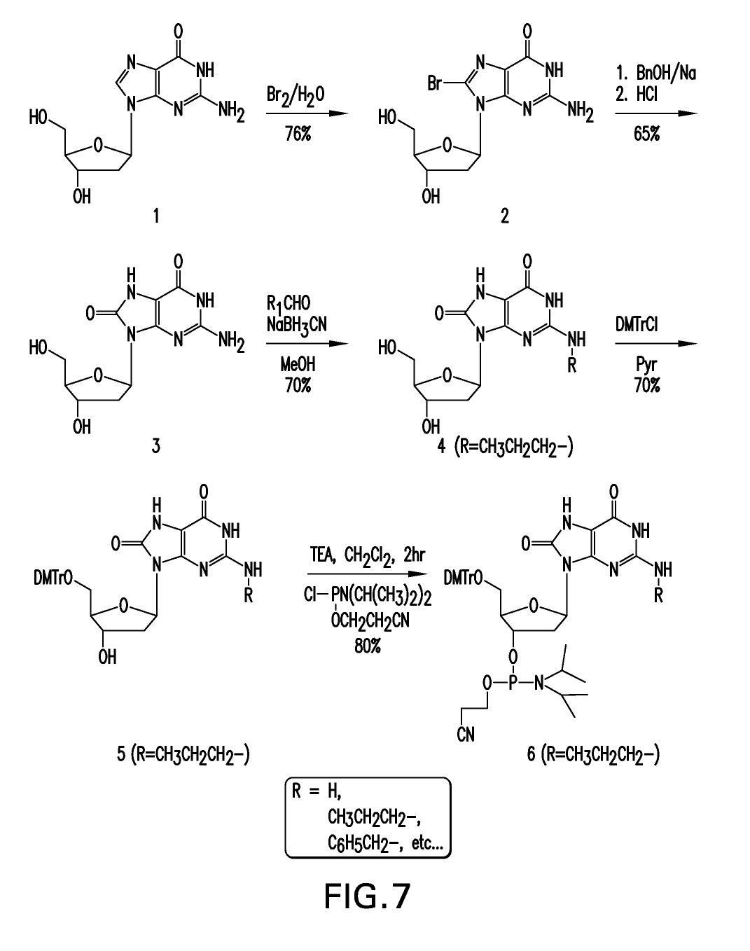

Preliminary studies indicate that the 8-oxodG (no alkyl group) is well accommodated in antisense strands and these strands also possess knockdown capabilities (FIG. 6). These studies indicate that 8-oxodG can be used as a switch and form Watson-Crick (anti) pairing and Hoogsten pairing (syn) while binding with the sense strand and mRNA was not compromised. In FIG. 6, Anti=5′-CAGUUUCUCUUGCAUUUCCtt-3′ (SEQ ID NO: 3); Anti-1=5′-CAGUUUCUCUUGCAUXUCCtt-3 (SEQ ID NO: 4); Anti-2=5′-CAGUUUCUCUXGCAUUUCCtt-3 (SEQ ID NO: 5); and Anti-3=5′-CAGXUUCUCUUGCAUUUCCtt-3 (SEQ ID NO: 6), wherein X=8-oxo-dG.

Replacement of Us with N²-alkyl-8-oxodG or N²-alkyl-8-oxoG can probe the stability of the syn:anti base pairs in different sequence contexts (FIG. 6, U₄ vs. U₁₁ vs. U₁₆), as well as the ability of siRNAs containing these base modifications to function in RNA interference.

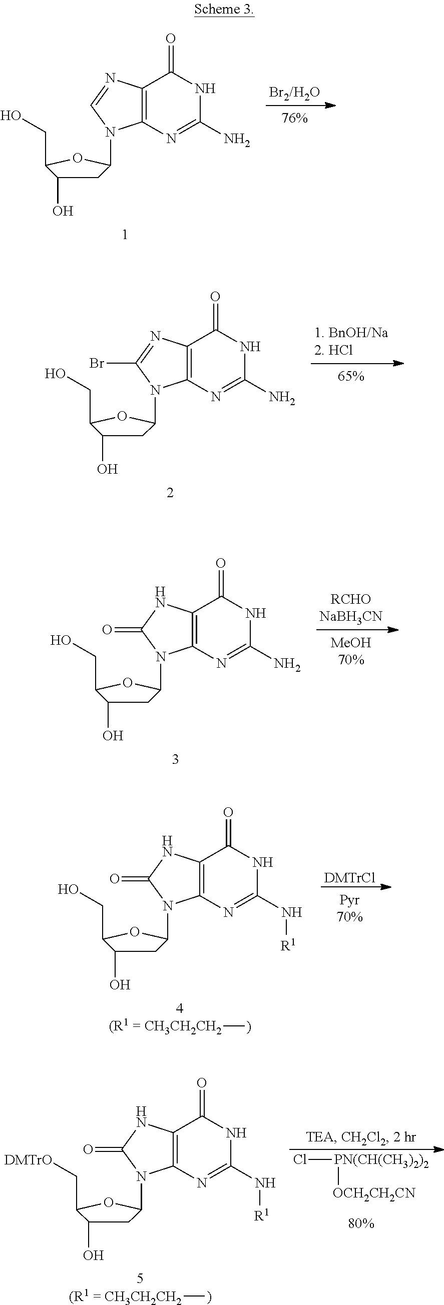

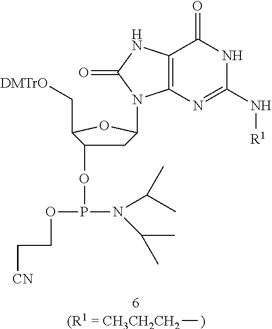

### Preparation of N²-n-propyl- 8-oxo-2′-deoxyguanosine phosphoramidite

An exemplary embodiment of a disclosed nucleobase was prepared according to Scheme 3. All chemicals are obtained commercially and used as received unless otherwise mentioned. The DMSO was dried over CaH₂, decanted, and distilled prior to use. Pyridine and CH₂Cl₂ were heated at reflux over CaH₂ and then distilled. Triethylamine was heated with Na pieces for 6 h, decanted, and then distilled from CaH₂. Benzene and toluene were heated at reflux over P₂O₅ and then distilled. Solvents and liquid reagents were introduced by oven-dried micro syringes. Merck silica gel 60 F254 precoated plates were used for thin layer chromatography (TLC). Column chromatography was conducted using silica gel 150 (60-200 mesh). ¹H NMR and ¹³C NMR spectra were recorded at 300 MHz and 75 MHz respectively. All mass spectrometric analyses were performed on a Quattro II Micromass spectrometer. LC-MS analysis was carried out using ACES C18 3.0 mm×100 mm reverse phase column with CH₃CN:H₂O [gradient-5% to 100% CH₃CN in 20 minutes] as the eluting solvent.

### Synthesis of 8-bromo-2′-deoxyguanosine (2)

Two g (7.4 mmol) of compound (1) suspended in 11 mL of doubly distilled water was reacted with 60 mL of saturated bromine water. The latter was added in 5 mL aliquots with vigorous stirring of the reaction mixture, and the yellow color was allowed to fade between additions. The precipitated 8-bromoguanosine (2) was recovered by filtration, washed extensively with cold water, followed by cold acetone, and air-dried. Yield: 1.8 g (76%). All other physical and spectroscopic data was identical to that previously reported (Luo et al. 2000).

### Synthesis of 8-oxo-2′-deoxyguanosine (3)

This compound was synthesized as previously described (Bodepudi et al. 1992). Briefly, DMSO (35 mL) was added to a solution of sodium benzoylate [from freshly distilled benzyl alcohol (14 mL, 130 mmol) with sodium (400 mg) at 60° C. until the solution was homogeneous under nitrogen]. To the resulting mixture was added 2 (1.8 g, 5.2 mmol) in DMSO (15 mL), and the mixture was heated at 65° C. for 24 h and then cooled to room temperature. After that DMSO was removed by vacuum distillation. Then the column purification yielded 8-(Benzyloxy)-2′-deoxyguanosine which on treatment with 1 M HCl yielded the required product 3. Yield: 950 mg (65%). All other physical and spectroscopic data was identical to that previously reported (Bodepudi et al. 1992).

### N²-n-Propyl-3′,5′-di-O-acetyl-8-oxo-2′-deoxyguanosine⁴(4)

To a suspension of the 8-oxo-2′-deoxyguanosine (950 mg, 3.3 mmol) and NaBH₃CN (628 mg, 8.8 mmol) in methanol (80 mL) was added propanaldehyde (6.7 mL, 114 mmol) in one portion, and the mixture was heated at 50° C. for overnight under nitrogen. After removal of the solvent under reduced pressure, the residue was purified by column chromatography to yield 762 mg (70%) of crude N²-n-Propyl-8-oxo-2′-deoxyguanosine (4). The structure was characterized using NMR and Mass spectrometry.

### 7,8-Dihydro-5′-O-4,4′-dimethoxytrityl- N²-n-Propyl-8-oxo-2′-deoxyguanosine (5)

To a solution of 6 (200 mg, 0.6 mmol) in dry pyridine (6 mL) cooled in ice-water was added 4,4′-DMTrCl (250 mg, 0.7 mmol). The cooling bath was then removed and stirring was continued at room temperature for 15 min. The reaction mixture was cooled in ice water and quenched with water (50 mL). The solution was extracted with CH₂Cl₂ (5×20 mL), and the combined organic layers were washed with H₂O (2×20 mL) and then dried over MgSO₄. The solvent was evaporated under reduced pressure, and the crude residue was purified by chromatography to yield 270 mg (70%). The structure was characterized using NMR and Mass spectrometry.

### 3′-O-[(Diisopropylamino)-(2-cyanoethoxy)phosphino]-7,8-dihydro-5′-O-(4,4′-dimethoxytrityl)-8-oxo-2′-deoxyguanosine (6)

To a mixture of 7 (270 mg, 0.4 mmol) dried over P₂O₅ in a vacuum desiccator for 24-48 hr and then co-evaporated with a mixture of dry CH₂Cl₂ and benzene prior to reaction) and dry Et₃N (100 mg, 1 mmol) in dry CH₂Cl₂ (1 mL) under N₂ was added 2-cyanoethyl N,N-diisopropylphosphoramidochloridite (120 mg, 0.5 mmol). The progress of the reaction was monitored by TLC analysis. Once the starting material disappears completely the reaction mixture is evaporated and a column chromatography yields 250 mg (70%). The structure was characterized using NMR and Mass spectrometry.

The phosphoramidites were then incorporated into oligomers at specific positions (4, 11 and 16 of antisense strands) using DNA/RNA synthesizer. The crude oligomers were then deprotected by treating with conc. ammonium hydroxide with 0.25 mM of 2-mercaptoethanol (to prevent further oxidation of 8-oxo dG). The deprotected oligomers were purified using HPLC and then characterized using electro spray mass spectrometry (ESI/MS) using a Quattro II mass spectrometer. The purified sense and antisense strands were hybridized by heating equimolar quantities to 95° C. for 5 min and cooling back to room temperature in the presence of Tris buffer at pH 7.4. Duplex RNAs were then quantified by UV-Visible spectroscopy.

### Example 2

### siRNA-Mediated Gene Knockdown

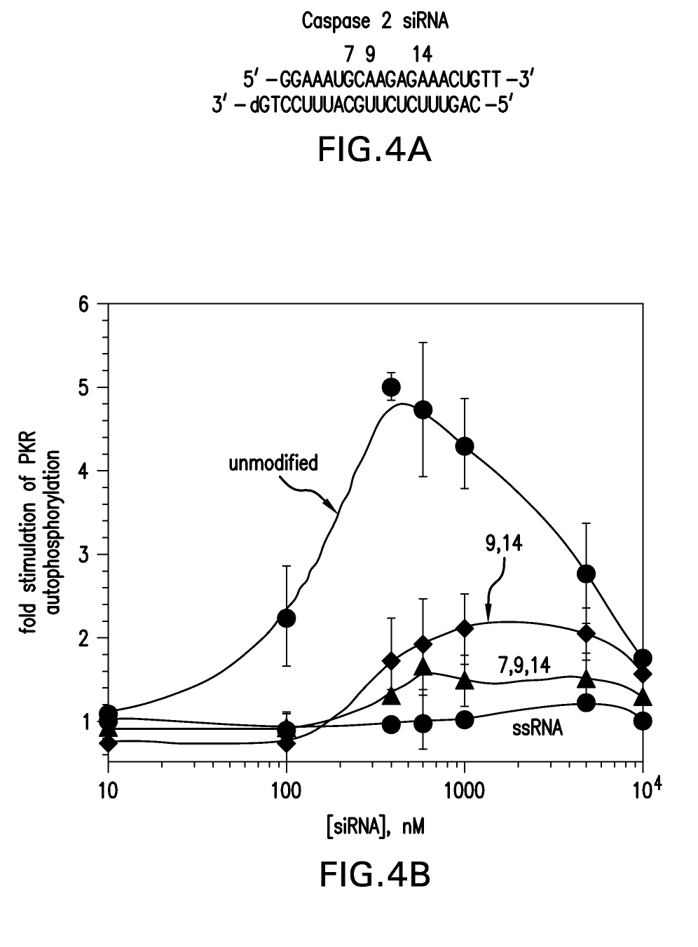

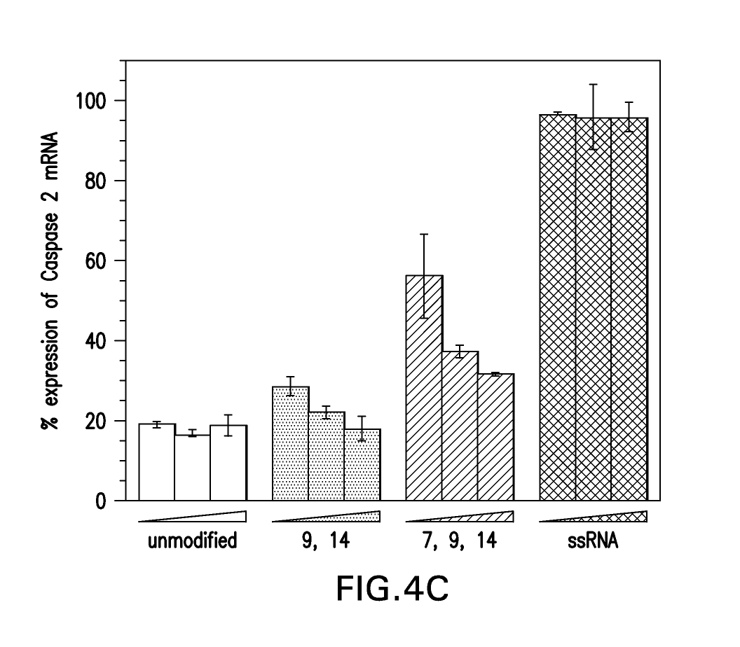

Caspase-2 is one of the cysteine-aspartate proteases that play critical roles in the initiation and execution of apoptosis (Zhivotovsky et al. 2005). The knock-down of caspase 2 can have research applications including but not limited to being able to sustain cells for characterization of various cellular functions. The knock-down of other key proteins for cell survival can also have research and clinical applications. In the present example, the modified base is introduced into siRNA targeting knock down of caspase 2. In a set of experiments, the knock down studies utilized siRNA have modifications at positions 7, 9, and 14 or modifications at positions 9 and 14). (FIG. 4). N² benzyl modification of nucleotides near sense strand positions 7, 9, and 14 blocked binding to the four dsRBM binding sites identified. These modified interfering RNAs show reduced binding with dsRBMs, and also knocked down the desired target gene in a dose dependent manner (Puthenveetil et al. 2006) (FIG. 4). All of the modified guanosines show efficient knock-down at 1 nM concentrations. These modified bases are effective in the siRNA pathway and increase the efficacy of inhibition of gene expression while exhibiting fewer off-target effects. These studies indicated that the N² alkyl substitution facing the minor groove prevents its interaction with dsRBM containing proteins while maintaining its ability to knock down the gene.

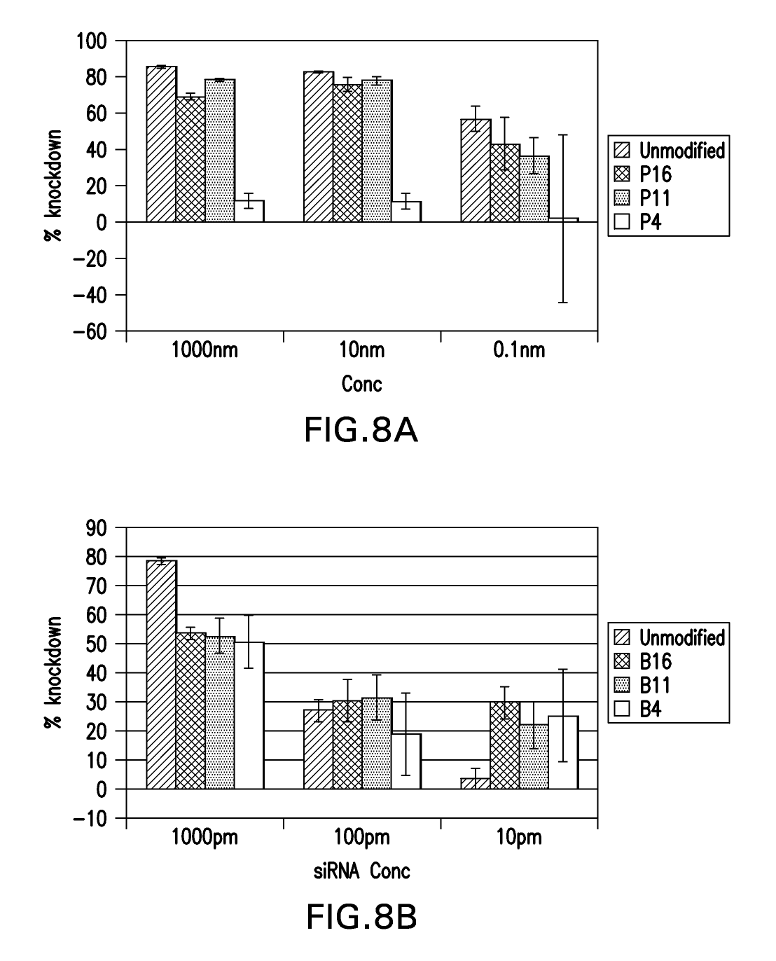

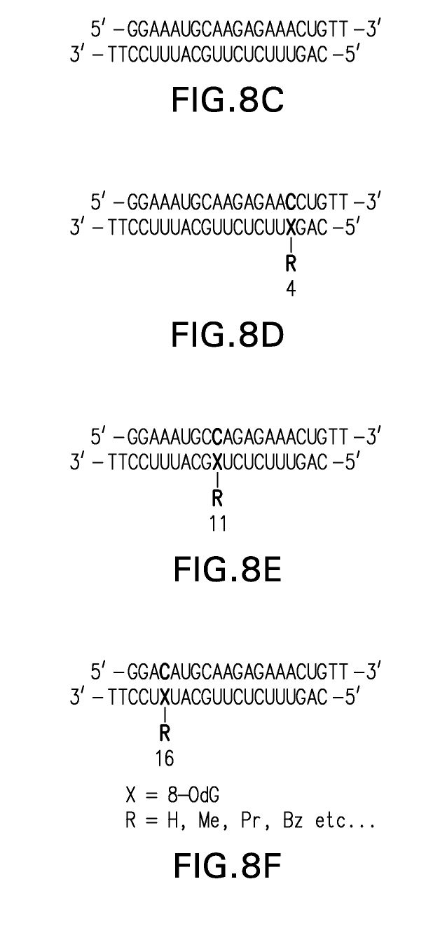

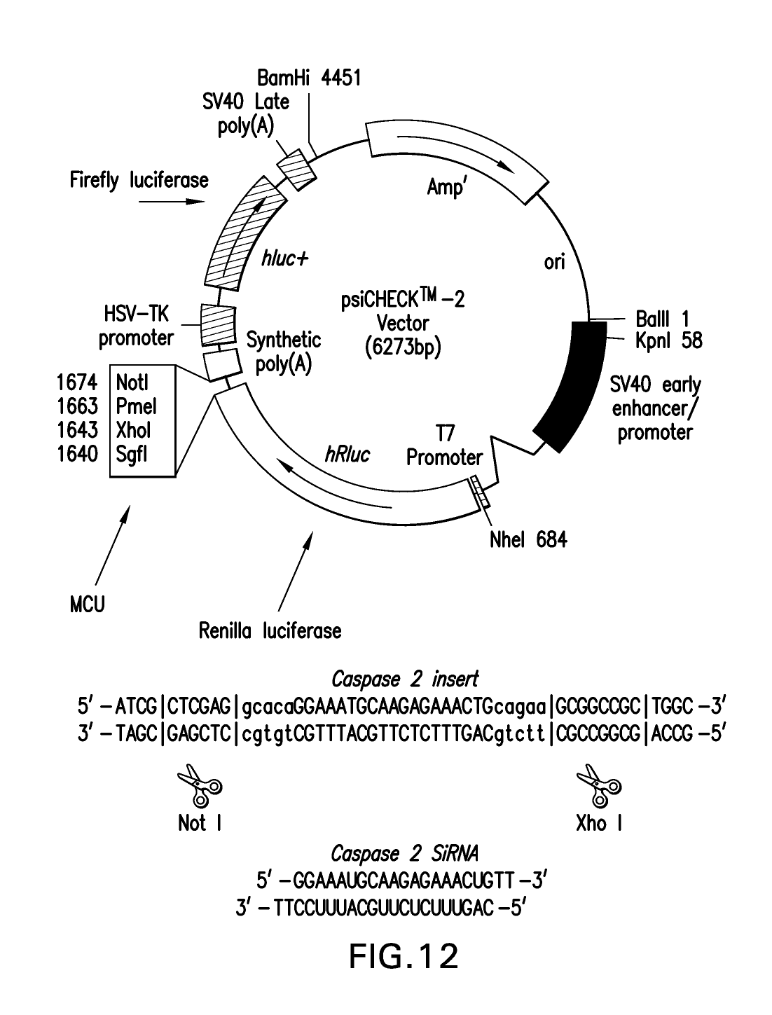

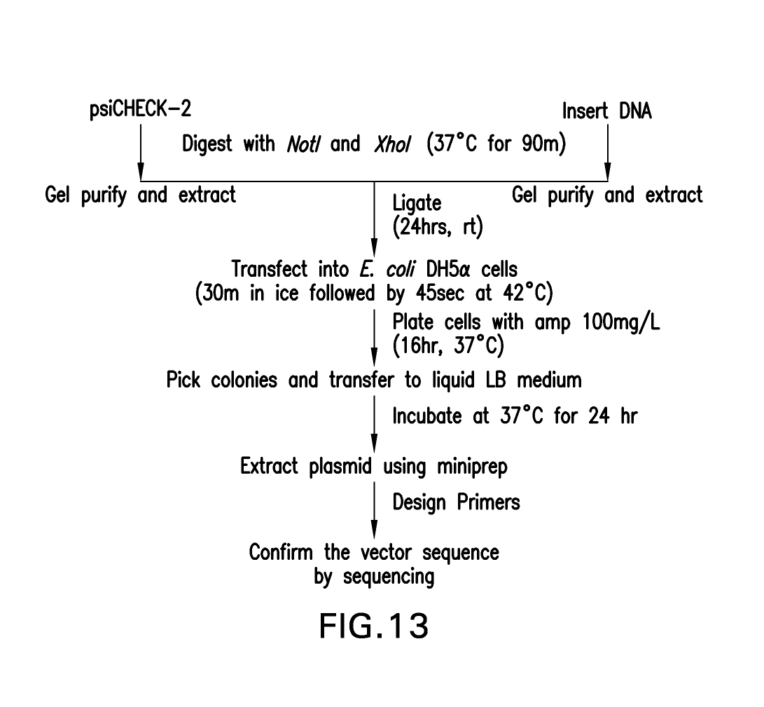

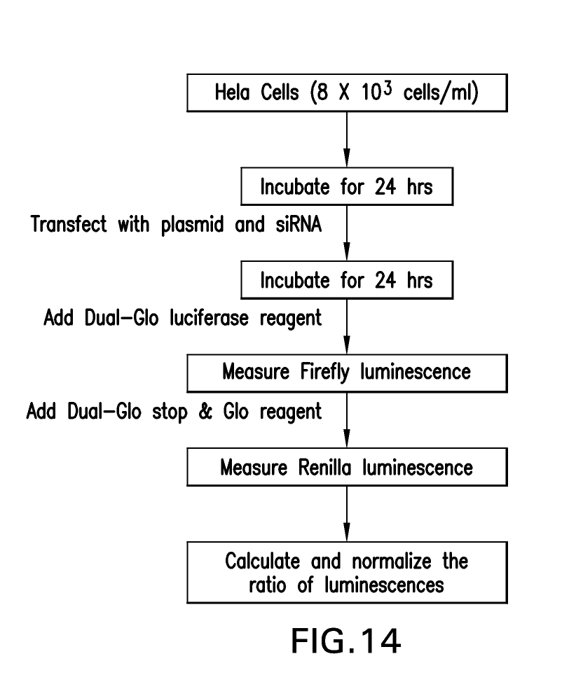

In another experiment, HeLa cells (8000 cells per well) were grown in 96-well plates for 24 hrs and after 24 hrs were transfected with 0.1-100 nM siRNA, 40 ng of Caspase-2-psiCHECK™-2 Vector (caspase-2 gene is cloned into psiCHECK™-2 [Promega, Madison, Wis.] using NotI and XhoI) using 0.5 mL siPORT NeoFX [Ambion, Austin, Tex.] per well. (See FIG. 12 and FIG. 13). Caspase 2 siRNA (FIG. 8) that is well optimized to knock down caspase 2 gene is used as the positive control. After 24 hrs, the gene knockdown is measured using the Dual-Glo® Luciferase Assay System [Promega, Madison, Wis.]. (See FIG. 14). The normalized results of the experiments are shown in FIG. 8. Results show that siRNAs containing the modified bases mostly retains the gene knockdown ability in comparison to unmodified siRNA. Preliminary studies (Puthenveetil et al. 2006) indicate that the N²-alkyl 2′-deoxyguanosines have reduced protein binding and hence increased efficacy. These experiments indicate that the currently synthesized analogues also have less protein binding.

In FIG. 8C, the unmodified caspase 2 siRNA sense strand is 5′-GGAAAUGCAAGAGAAACUGTT-3′ (SEQ ID NO: 7) and the anti-sense strand is 3′-TTCCUUUACGUUCUCUUUGAC-5′ (SEQ ID NO: 8). In FIG. 8D, the caspase 2 siRNA sense strand is 5′-GGAAAUGCAAGAGAACCUGTT-3 (SEQ ID NO: 9) and the anti-sense strand modified at position 4 is 3′-TTCCUUUACGUUCUCUUXGAC-5′ (SEQ ID NO: 10). In FIG. 8E, the caspase 2 siRNA sense strand is 5′-GGAAAUGCCCAGAGAAACUGTT-3′ (SEQ ID NO: 11) and the anti-sense strand modified at position 11 is 3′-TTCCUUUACGXUCUCUUUGAC-5′ (SEQ ID NO: 12). In FIG. 8F, the caspase 2 siRNA sense strand is 5′-GGACAUGCAAGAGAAACUGTT-3′ (SEQ ID NO: 13) and the anti-sense strand modified at position 16 is 3′-TTCCUXUACGUUCUCUUUGAC-5′ (SEQ ID NO: 14). In FIGS. 8C-8F, X is 8-OdG and R is H, Me, Pr. Bz, etc.

FIG. 12 shows the 5′ strand for the caspase 2 insert, wherein the 5′ strand reads 5′-ATCGCTCGAGgcacaGGAAATGCAAGAGAAACTGcagaaGCGGCCGCTGGC-3′ (SEQ ID NO: 16) and the 3′ strand reads 3′-TAGCGAGCTCcgtgtCGTTTACGTTCTCTTTGACgtcttCGCCGGCGACCG-5′ (SEQ ID NO: 17) (the Not1 and Xho1 restriction sites are underlined). Also in FIG. 12, the 5′ strand of caspase 2 siRNA reads 5′-GGAAAUGCAAGAGAAACUGTT-3′ (SEQ ID NO: 18) and the 3′ strand reads 3′-TTCCUUUACGUUCUCUUUGAC-5′ (SEQ ID NO: 19).

### Example 3

### Stability of Modified siRNA Molecules

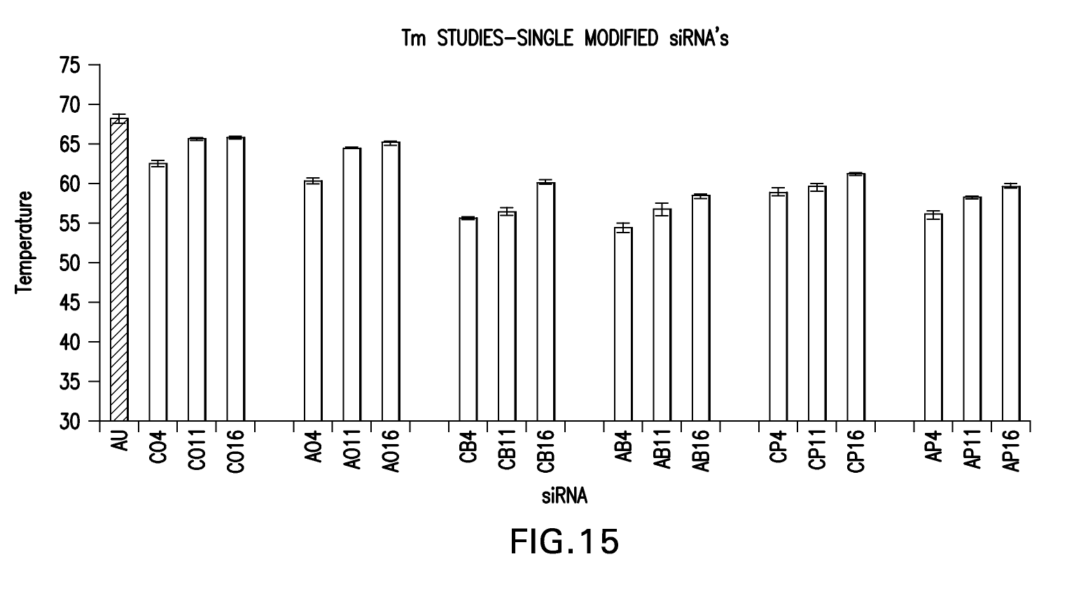

The effect of 8-oxo-2′-deoxyguanosine (8-oxo-dG) and N²-alkyl substitutions of 8-oxo-2′-deoxyguanosine on duplex stability was investigated via thermal denaturation (T_{M}) studies of a RNA duplex. The nucleoside analogs were incorporated into the duplex at three different positions (4, 11 and 16) from 5′ end of antisense strand opposite adenine (A), and guanine (G) The modified strands containing 8-oxodG and its analogues were also compared with the unmodified strand (AU) containing A:U base pair. (FIG. 15).

The T_{M} experiments were performed with duplexes that were formed by hybridizing 1 nmol complementary strands in 1 mL buffer (10 mM Tris-HCl, pH 7.5, with 100 mM NaCl). The solution was heated at 95° C. for 5 min and allowed to cool slowly over a period of 12 hours to room temperature. These duplexes were directly used in T_{M} analyses. T_{M} experiments were performed on a Beckmann DU 7400 spectrophotometer with a multi-cuvette temperature controller. Duplexes (325 μL) were denatured in triplicate over a temperature range of 25° C. to 80° C. at 0.5° C./min. The absorbance at 260 nm was recorded every 0.5° C. The fraction of oligonucleotides in a duplex (f) was determined by fitting the data to the equation:

$$f = \frac{A - {A}_{ss}}{{A}_{ds} - {A}_{ss}}$$

where A=Absorbance of sample at each temperature, Ads=Absorbance of double stranded oligo, and Ass=Absorbance of single stranded oligo. The f vs. temperature was graphed for the linear portion of the curve (range of values where f˜0.4-0.6). A linear regression was performed and the T_{M} was determined from the point on the line where f=0.5. The values reported represent the average of three experiments. The error bars on the graph and the ±values in the manuscript indicate ±standard deviation.

The T_{M} of the unmodified strand (AU) was 68+0.5° C. (FIG. 15). On introduction of OG:C at 16, and 11 positions resulted in decrease in T_{M} by ˜2° C., whereas at 4 position it decreased by 5° C. indicating the high sensitivity to modification at position 4. The introduction of OG: A at the same positions showed further decrease in T_{M} indicating that the OG:A was less stable in comparison with OG:C. Other analogues of 8-oxo-dG such as propyl and benzyl showed similar T_{M} patterns, among these benzyl modification was more destabilizing than propyl analogue or 8-oxo-dG itself The order of stability of these oligos was unmodified >8-oxo-dG>propyl>benzyl. (See Tables 2-4). The increase in bulkiness of N² group lead to decreased stability of the oligos. Though there was slight decrease in T_{M}, all the oligos still showed high T_{M} (above 60° C.) indicating that the modifications were well tolerated at these positions. The small difference in T_{M} between the pairing against A and C indicates that 8-oxo-dG can switch itself according to the complementary base and pair with and C with almost equal stability. Furthermore, these data indicate that substituents with a range of size and structure into the minor groove when pairing with C can be projected, and can be used to study minor groove contacts at A:U sites and to disrupt complexes with dsRBMs. (See Tables 2-4).

In FIG. 15, which shows the T_{M} studies for singly-modified siRNAs, (1) AU=unmodified siRNA; (2) CO4, CO11, CO16=antisense strand modification with 8-Odg at 4, 11 and 16 and pairs against C in sense strand; (3) AO4, AO11, AO16=antisense strand modification with 8-Odg at 4, 11 and 16 and pairs against A in sense strand; (4) CB4, CB11, CB16=antisense strand modification with 8-Oxo-2-benzyldg at 4, 11 and 16 and pairs against C in sense strand; (5) AB4, AB11, AB16=antisense strand modification with 8-Oxo-2-benzyldg at 4, 11 and 16 and pairs against A in sense strand; (6) CP4, CP11, CP16=antisense strand modification with 8-Oxo-2-propyldg at 4, 11 and 16 and pairs against C in sense strand; and (7) AP4, AP11, AP16=antisense strand modification with 8-Oxo-2-propyldg at 4, 11 and 16 and pairs against A in sense strand.

TABLE 2
Stability of Singly Modified siRNAs with 8-ODG

| | TM | TM | |
|---|---|---|---|
| siRNA | (G*:C) | (G*:A) | Sequence |
| AU | 68.2 ± 0.53 | 68.2 ± 0.53 | 5′-GGAAAUGCAAGAGAAACUGTT-3′ (SEQ ID NO: 20) |
| | | | 3′-TTCCUUUACGUUCUCUUUGAC-5′ (SEQ ID NO: 21) |
| O16 | 65.9 ± 0.19 | 65.2 ± 0.29 | 5′-GGACAUGCAAGAGAAACUGTT-3′ (SEQ ID NO: 22) |
| | | | 3′-TTCCUXUACGUUCUCUUUGAC-5′ (SEQ ID NO: 23) |
| O11 | 65.7 ± 0.25 | 64.5 ± 0.09 | 5′-GGAAAUGCCAGAGAAACUGTT-3′ (SEQ ID NO: 24) |
| | | | 3′-TTCCUUUACGXUCUCUUUGAC-5′ (SEQ ID NO: 25) |
| O4 | 62.2 ± 0.32 | 60.5 ± 0.38 | 5′-GGAAAUGCAAGAGAACCUGTT-3′ (SEQ ID NO: 26) |
| | | | 3′-TTCCUUUACGUUCUCUUXGAC-5′ (SEQ ID NO: 27) |

X = 8-ODG

TABLE 3
Stability of Singly Modified siRNAs with N²-Propyl-8-ODG

| | T_{M} | T_{M} | |
|---|---|---|---|
| siRNA | (G*:C) | (G*:A) | Sequence |
| AU | 68.2 ± 0.53 | 68.2 ± 0.53 | 5′-GGAAAUGCAAGAGAAACUGTT-3′ (SEQ ID NO: 28) |
| | | | 3′-TTCCUUUACGUUCUCUUUGAC-5′ (SEQ ID NO: 29) |
| P16 | 61.3 ± 0.24 | 59.9 ± 0.24 | 5′-GGACAUGCAAGAGAAACUGTT-3′ (SEQ ID NO: 30) |
| | | | 3′-TTCCUXUACGUUCUCUUUGAC-5′ (SEQ ID NO: 31) |
| P11 | 59.7 ± 0.47 | 58.4 ± 0.18 | 5′-GGAAAUGCCAGAGAAACUGTT-3′ (SEQ ID NO: 32) |
| | | | 3′-TTCCUUUACGXUCUCUUUGAC-5′ (SEQ ID NO: 33) |
| P4 | 59.0 ± 0.44 | 56.2 ± 0.54 | 5′-GGAAAUGCAAGAGAACCUGTT-3′ (SEQ ID NO: 34) |
| | | | 3′-TTCCUUUACGUUCUCUUXGAC-5′ (SEQ ID NO: 35) |

X = N²-Propyl-8-ODG

TABLE 4
Stability of Singly Modified siRNAs with N²-Benzyl-8-ODG

| | T_{M} | T_{M} | |
|---|---|---|---|
| siRNA | (G*:C | (G*:A) | Sequence |
| AU | 68.2 ± 0.53 | 68.2 ± 0.53 | 5′-GGAAAUGCAAGAGAAACUGTT-3′ (SEQ ID NO: 36) |
| | | | 3′-TTCCUUUACGUUCUCUUUGAC-5′ (SEQ ID NO: 37) |
| B16 | 60.3 ± 0.25 | 58.6 ± 0.33 | 5′-GGACAUGCAAGAGAAACUGTT-3′ (SEQ ID NO: 38) |
| | | | 3′-TTCCUXUACGUUCUCUUUGAC-5′ (SEQ ID NO: 39) |
| B11 | 56.5 ± 0.50 | 56.9 ± 0.74 | 5′-GGAAAUGCCAGAGAAACUGTT-3′ (SEQ ID NO: 40) |
| | | | 3′-TTCCUUUACGXUCUCUUUGAC-5′ (SEQ ID NO: 41) |
| B4 | 55.7 ± 0.25 | 54.6 ± 0.57 | 5′-GGAAAUGCAAGAGAACCUGTT-3′ (SEQ ID NO: 42) |
| | | | 3′-TTCCUUUACGUUCUCUUXGAC-5′ (SEQ ID NO: 43) |

X = N²-Benzyl-8-ODG

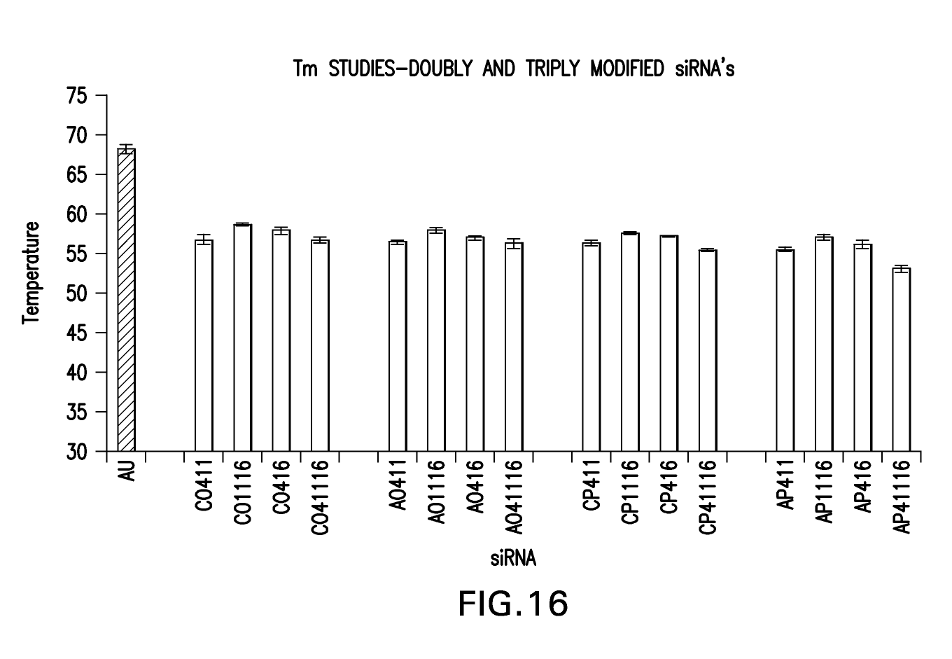

The nucleoside analogs were incorporated into the duplex at more than one position (4, 11), (11, 16), (4, 16) (4, 11, 16) of the antisense strands opposite adenine (A), and guanine (G). (FIG. 16). Introduction of the modifications at more than one position decreased the T_{M} further. Most of the T_{M} values of strands having modifications at more than one position were around or above 55° C. indicating that the strands formed a stable duplex. Further, the difference in T_{M} between pairing against guanine and adenine was marginal indicating that the stable duplexes can be formed by 8-oxodG against A or G.

In FIG. 16, (1) AU=unmodified siRNA; (2) CO411, CO1116, CO416, CO41116=antisense strand modifications with 8-oxo-dG at (4, 11), (11, 16), (4,16) (4,11,16) and pairs against C in sense strand; (3) AO411, AO1116, AO416, AO41116=antisense strand modifications with 8-oxo-dG at (4, 11), (11, 16), (4,16) (4,11,16) and pairs against A in sense strand; (4) CP411, CP1116, CP416, CP41116=antisense strand modifications with 8-oxo-2-propyldG at (4, 11), (11,16), (4,16) (4,11,16) and pairs against C in sense strand; (5) AP411, AP1116, AP416, AP41116=antisense strand modifications with 8-Oxo-2-propyldG at (4,11), (11,16), (4,16) (4,11,16) and pairs against A in sense strand. (See Tables 5-6).

TABLE 5
Stability of Multiply Modified siRNAs with 8-ODG Modifications

| | T_{M} | T_{M} | |
|---|---|---|---|
| siRNA | (G*:C | (G*:A) | Sequence |
| AU | 68.2 ± 0.53 | 68.2 ± 0.53 | 5′-GGAAAUGCAAGAGAAACUGTT-3′ (SEQ ID NO: 44) |
| | | | 3′-TTCCUUUACGUUCUCUUUGAC-5′ (SEQ ID NO: 45) |
| O114 | 56.8 ± 0.59 | 56.5 ± 0.27 | 3′-TTCCUUUACGXUCUCUUXGAC-5′ (SEQ ID NO: 46) |
| O1611 | 58.7 ± 0.24 | 57.9 ± 0.37 | 3′-TTCCUXUACGXUCUCUUUGAC-5′ (SEQ ID NO: 47) |
| O164 | 57.9 ± 0.45 | 57.0 ± 0.22 | 3′-TTCCUXUACGUUCUCUUXGAC-5′ (SEQ ID NO: 48) |
| O16114 | 56.7 ± 0.38 | 56.3 ± 0.56 | 3′-TTCCUXUACGXUCUCUUXGAC-5′ (SEQ ID NO: 49) |

X = 8-ODG

TABLE 6
Stability of Multiply Modified siRNAs with N²-Propyl-8-ODG Modifications

| | T_{M} | T_{M} | |
|---|---|---|---|
| siRNA | (G*:C) | (G*:A) | Sequence |
| AU | 68.2 ± 0.53 | 68.2 ± 0.53 | 5′-GGAAAUGCAAGAGAAACUGTT-3′ (SEQ ID NO: 50) |
| | | | 3′-TTCCUUUACGUUCUCUUUGAC-5′ (SEQ ID NO: 51) |
| P114 | 56.4 ± 0.32 | 55.5 ± 0.29 | 3′-TTCCUUUACGXUCUCUUXGAC-5′ (SEQ ID NO: 52) |
| P1611 | 57.6 ± 0.19 | 57.0 ± 0.34 | 3′-TTCCUXUACGXUCUCUUUGAC-5′ (SEQ ID NO: 53 |
| P164 | 57.2 ± 0.09 | 56.2 ± 0.49 | 3′-TTCCUXUACGUUCUCUUXGAC-5′ (SEQ ID NO: 54) |
| P16114 | 55.4 ± 0.12 | 53.1 ± 0.50 | 3′-TTCCUXUACGXUCUCUUXGAC-5′ (SEQ ID NO: 55) |

X = N²-Propyl-8-ODG

### Example 4

### PKR Binding Studies

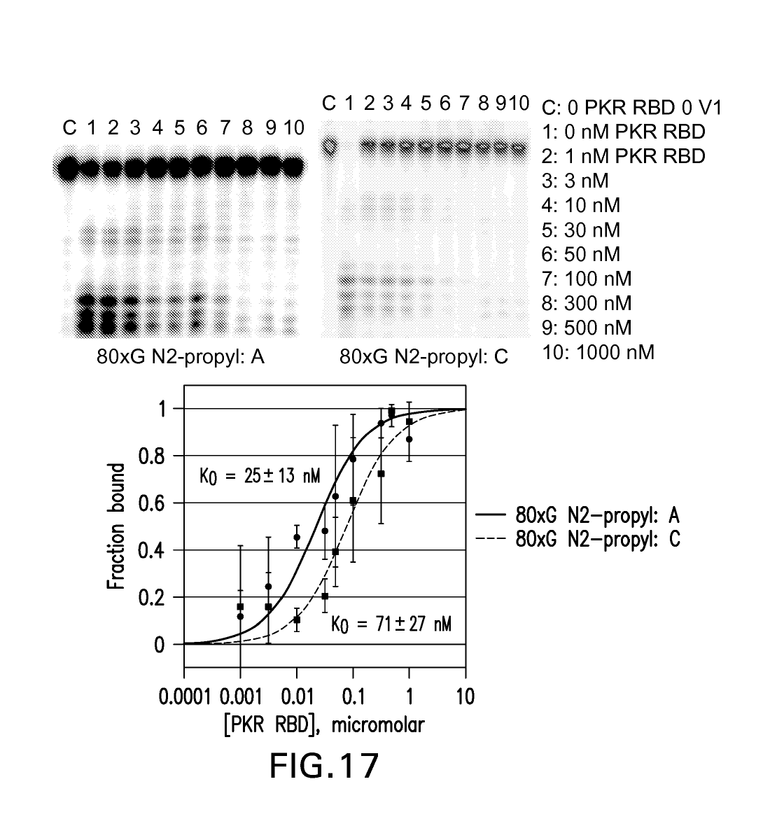

The sterically bulky functional groups at N²-8-oxo-2′-deoxyguanosine prevented binding with double strand RNA binding motif (dsRBM) containing proteins such as PKR. Ribonuclease V1 foot printing was used to measure binding affinities for the RNA-binding domain (RBD) of PKR for each duplex. Duplexes containing N²-propyl-8-oxo-2′-deoxyguanosine paired against A and C at three different positions were used. V1 nuclease was a duplex-specific cleaver so if RNA-binding domain of PKR binds with duplex RNA, then V1 nuclease cannot cleave the duplex siRNA. As PKR RBD watitrated into the sample, the cleavage bands induced by V1 were inhibited due to increased PKR binding with duplex siRNA. FIG. 17 shows two duplexes with three 8-Oxo-dG-N² propyl modifications against sense strands with 3 A's and C's. The N²-propyl-8-oxo-2′-deoxyguanosine pairing with C placed bulk in the minor groove and prevented the dsRBM proteins like PKR, but pairing with A hide the group in the major groove away from where dsRBMs binded and increased PKR binding. These experiments showed that the N²-propyl-8-oxo-2′-deoxyguanosine pairing with C (as in case with sense-antisense pairing) binding with dsRBM containing proteins was reduced, whereas N²-propyl-8-oxo-2′-deoxyguanosine pairing with A (as in case of mRNA-antisense pairing) cleaving was not affected. The difference was around 3 fold with propyl side chain. Ribonuclease V1 footprinting was used to measure binding affinities for the RNA-binding domain of PKR for each duplex. The Kd's reported in the FIG. 17 were the average of three independently fitted data sets with standard deviations.

What is claimed is:

1. A compound of Formula I: 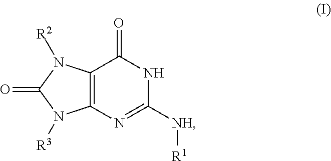
wherein R¹ is:
i) substituted or unsubstituted C₁-C₆ linear, branched, or cyclic alkyl;
ii) substituted or unsubstituted C₂-C₆ linear, branched, or cyclic alkenyl;
iii) substituted or unsubstituted C₂-C₆ linear or branched alkynyl;
iv) substituted or unsubstituted C₆-C₁₀ aryl;
v) substituted or unsubstituted C₁-C₉ heteroaryl; or
vi) substituted or unsubstituted C₁-C₉ heterocyclic;
provided that R¹ does not comprise pyrenyl, 1-oxopropyl, or tetrahydrofuranyl;
wherein R² is:
i) substituted or unsubstituted C₁-C₆ linear, branched, or cyclic alkyl;
ii) substituted or unsubstituted C₂-C₆ linear, branched, or cyclic alkenyl;
iii) substituted or unsubstituted C₂-C₆ linear or branched alkynyl;
iv) substituted or unsubstituted C₆-C₁₀ aryl;
v) substituted or unsubstituted C₁-C₉ heteroaryl;
vi) substituted or unsubstituted C₁-C₉ heterocyclic; or
vii) hydrogen;
wherein R³ is
i) substituted or unsubstituted C₁-C₆ linear, branched, or cyclic alkyl;
ii) substituted or unsubstituted C₂-C₆ linear, branched, or cyclic alkenyl;
iii) substituted or unsubstituted C₂-C₆ linear or branched alkynyl;
iv) substituted or unsubstituted C₆-C₁₀ aryl;
v) substituted or unsubstituted C₁-C₉ heteroaryl;
vi) substituted or unsubstituted C₁-C₉ heterocyclic; or
vii) hydrogen.

2. The compound of claim 1, wherein R¹ is substituted or unsubstituted methyl, ethyl, n-propyl, iso-propyl, n-butyl, iso-butyl, sec-butyl, tent-butyl, or benzyl.

3. The compound of claim 1, wherein R² is hydrogen.

4. The compound of claim 1, wherein R³ is substituted or unsubstituted tetrahydrofuranyl or tetrahydropyranyl.

5. The compound of claim 1, wherein R³ is a residue of Formula II: 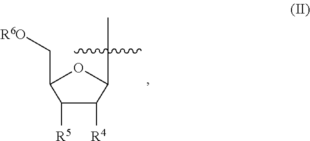
wherein R⁴ is:
i) hydrogen;
ii) hydroxyl;
iii) alkoxy;
iv) amino; or
v) halogen;
wherein R⁵ is:
i) hydrogen;
ii) hydroxyl;
iii) alkoxy;
iv) amino;
v) halogen;
vi) C₁-C₁₂ phosphonite, phosphate, phosphonate, or phosphoryl; or
vii) an O-linked solid support; and
wherein R⁶ is:
i) hydrogen;
ii) a protecting group;
iii) a monophosphate;
iv) a diphosphate;
v) a triphosphate;
vi) a nucleotide; or
vii) a deoxynucleotide.

6. The compound of claim 5, wherein R⁵ is:
i) —O—(N,N-diisopropyl O-methyl phosphoramidite); or
ii) —O—(N,N-diisopropyl O-2-cyanoethyl phosphoramidite).

7. The compound of claim 5, wherein R⁶ is:
i) dimethoxytrityl (DMT);
ii) monomethoxytrityl;
iii) 9-phenylxanthen-9-yl (Pixyl); or
iv) 9-(p-methoxyphenyl)xanthen-9-yl (Mox).

8. A nucleoside of Formula III: 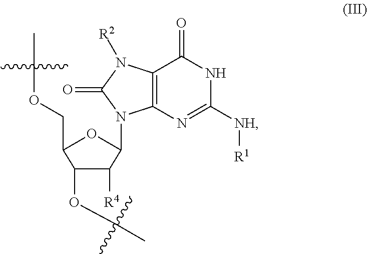
wherein R¹ is:
i) substituted or unsubstituted C₁-C₆ linear, branched, or cyclic alkyl;
ii) substituted or unsubstituted C₂-C₆ linear, branched, or cyclic alkenyl;
iii) substituted or unsubstituted C₂-C₆ linear or branched alkynyl;
iv) substituted or unsubstituted C₆-C₁₀ aryl;
v) substituted or unsubstituted C₁-C₉ heteroaryl; or
vi) substituted or unsubstituted C₁-C₉ heterocyclic;
provided that R¹ does not comprise pyrenyl, 1-oxopropyl, or tetrahydrofuranyl;
wherein R² is:
i) substituted or unsubstituted C₁-C₆ linear, branched, or cyclic alkyl;
ii) substituted or unsubstituted C₂-C₆ linear, branched, or cyclic alkenyl;
iii) substituted or unsubstituted C₂-C₆ linear or branched alkynyl;
iv) substituted or unsubstituted C₆-C₁₀ aryl;
v) substituted or unsubstituted C₁-C₉ heteroaryl;
vi) substituted or unsubstituted C₁-C₉ heterocyclic; or
vii) hydrogen; and
wherein R⁴ is:
i) hydrogen;
ii) hydroxyl;
iii) alkoxy;
iv) amino; or
v) halogen.

9. A method for making an alkylated compound, comprising, alkylating the amino group at position 6 of a compound of Formula IV, 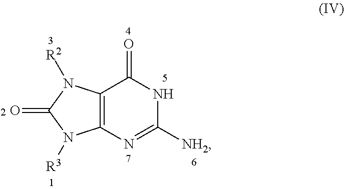
resulting in an alkylated compound of Formula V: 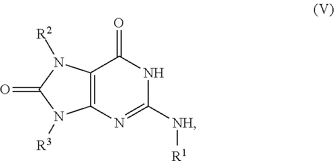
wherein R¹ is:
i) substituted or unsubstituted C₁-C₆ linear, branched, or cyclic alkyl;
ii) substituted or unsubstituted C₂-C₆ linear, branched, or cyclic alkenyl;
iii) substituted or unsubstituted C₂-C₆ linear or branched alkynyl;
iv) substituted or unsubstituted C₆-C₁₀ aryl;
v) substituted or unsubstituted C₁-C₉ heteroaryl; or
vi) substituted or unsubstituted C₁-C₉ heterocyclic;
provided that R¹ does not comprise pyrenyl, 1-oxopropyl, or tetrahydrofuranyl;
wherein R² is
i) substituted or unsubstituted C₁-C₆ linear, branched, or cyclic alkyl;
ii) substituted or unsubstituted C₂-C₆ linear, branched, or cyclic alkenyl;
iii) substituted or unsubstituted C₂-C₆ linear or branched alkynyl;
iv) substituted or unsubstituted C₆-C₁₀ aryl;
v) substituted or unsubstituted C₁-C₉ heteroaryl;
vi) substituted or unsubstituted C₁-C₉ heterocyclic; or
vii) hydrogen; and
wherein R³ is
i) substituted or unsubstituted C₁-C₆ linear, branched, or cyclic alkyl;
ii) substituted or unsubstituted C₂-C₆ linear, branched, or cyclic alkenyl;
iii) substituted or unsubstituted C₂-C₆ linear or branched alkynyl;
iv) substituted or unsubstituted C₆-C₁₀ aryl;
v) substituted or unsubstituted C₁-C₉ heteroaryl;
vi) substituted or unsubstituted C₁-C₉ heterocyclic; or
vii) hydrogen.

10. The method of claim 9, wherein alkylating the amino group comprises reacting the compound of Formula IV with an aldehyde of formula R¹CHO,
wherein R¹ is:
i) substituted or unsubstituted C₁-C₆ linear, branched, or cyclic alkyl;
ii) substituted or unsubstituted C₂-C₆ linear, branched, or cyclic alkenyl;
iii) substituted or unsubstituted C₂-C₆ linear or branched alkynyl;
iv) substituted or unsubstituted C₆-C₁₀ aryl;
v) substituted or unsubstituted C₁-C₉ heteroaryl; or
vi) substituted or unsubstituted C₁-C₉ heterocyclic;
provided that R¹ does not comprise pyrenyl, 1-oxopropyl, or tetrahydrofuranyl.

11. The method of claim 9, wherein alkylating the amino group comprises reacting the compound of Formula IV with a compound of formula R¹X,
wherein R¹ is:
i) substituted or unsubstituted C₁-C₆ linear, branched, or cyclic alkyl;
ii) substituted or unsubstituted C₂-C₆ linear, branched, or cyclic alkenyl;
iii) substituted or unsubstituted C₂-C₆ linear or branched alkynyl;
iv) substituted or unsubstituted C₆-C₁₀ aryl;
v) substituted or unsubstituted C₁-C₉ heteroaryl; or
vi) substituted or unsubstituted C₁-C₉ heterocyclic;
provided that R¹ does not comprise pyrenyl, 1-oxopropyl, or tetrahydrofuranyl; and X is Br, I, F, or Cl.

12. An oligonucleotide or polynucleotide comprising at least one of Formula VI: 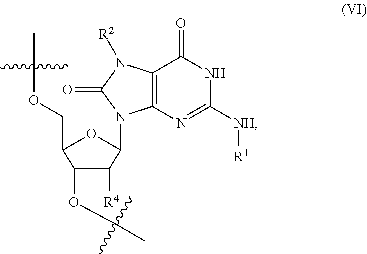
wherein R¹ is:
i) substituted or unsubstituted C₁-C₆ linear, branched, or cyclic alkyl;
ii) substituted or unsubstituted C₂-C₆ linear, branched, or cyclic alkenyl;
iii) substituted or unsubstituted C₂-C₆ linear or branched alkynyl;
iv) substituted or unsubstituted C₆-C₁₀ aryl;
v) substituted or unsubstituted C₁-C₉ heteroaryl; or
vi) substituted or unsubstituted C₁-C₉ heterocyclic;
provided that R¹ does not comprise pyrenyl, 1-oxopropyl, or tetrahydrofuranyl;
wherein R² is:
i) substituted or unsubstituted C₁-C₆ linear, branched, or cyclic alkyl;
ii) substituted or unsubstituted C₂-C₆ linear, branched, or cyclic alkenyl;
iii) substituted or unsubstituted C₂-C₆ linear or branched alkynyl;
iv) substituted or unsubstituted C₆-C₁₀ aryl;
v) substituted or unsubstituted C₁-C₉ heteroaryl;
vi) substituted or unsubstituted C₁-C₉ heterocyclic; or
vii) hydrogen; and
wherein R⁴ is:
i) hydrogen;
ii) hydroxyl;
iii) alkoxy;
iv) amino; or
v) halogen.

13. A method of blocking the binding of an off-target molecule to an siRNA molecule, comprising,
modifying at least one guanosine base of the siRNA molecule, and
administering to a subject the siRNA molecule.

14. The method of claim 13, wherein the siRNA molecule comprises two or more modified guanosine bases.

15. The method of claim 13, wherein the siRNA molecule comprises three or more modified guanosine bases.

16. The method of claim 13, wherein the modified guanosine base comprises the compound of claim 1.

17. The method of claim 13, wherein the off-target molecule is a double stranded RNA-binding motif (DSRBM).

18. The method of claim 17, wherein the DSRBM is RNA dependent protein kinase (PKR).

19. The method of claim 17, wherein the DSRBM is adenosine deaminase (ADAR).

20. The method of claim 13, wherein the off-target molecule is Toll-Like Receptor-7.

21. An siRNA molecule comprising at least one modified guanosine.

22. The siRNA molecule of claim 21, wherein the base opposite the modified guanosine is not complementary.

23. The method of claim 13, wherein the efficacy of the siRNA molecule is increased.

24. A kit comprising the compound of claim 1.

25. A kit comprising the compound of claim 5.

26. A kit comprising the nucleoside of claim 8.

27. A kit comprising the oligonucleotide of claim 12.

28. A kit comprising the polynucleotide of claim 12.

29. A set of nucleotides comprising at least one compound of claim 5.

30. A set of nucleotides comprising at least one oligonucleotide or polynucleotide of claim 12.


SEQUENCE LISTING (55 sequences)

SEQ ID NO: 1 - DNA, 21 bp, Artificial Sequence
  Description of Artificial Sequence Note = Synthetic Construct
ggaaaugcaagagaaacugtt

SEQ ID NO: 2 - DNA, 22 bp, Artificial Sequence
  Description of Artificial Sequence Note = Synthetic Construct
dgtccuuuacguucucuuugac

SEQ ID NO: 3 - DNA, 21 bp, Artificial Sequence
  Description of Artificial Sequence Note = Synthetic Construct
caguuucucuugcauuucctt

SEQ ID NO: 4 - DNA, 21 bp, Artificial Sequence
  Description of Artificial Sequence Note = Synthetic Construct
caguuucucuugcaunucctt

SEQ ID NO: 5 - DNA, 21 bp, Artificial Sequence
  Description of Artificial Sequence Note = Synthetic Construct
caguuucucungcauuucctt

SEQ ID NO: 6 - DNA, 21 bp, Artificial Sequence
  Description of Artificial Sequence Note = Synthetic Construct
cagnuucucuugcauuucctt

SEQ ID NO: 7 - DNA, 21 bp, Artificial Sequence
  Description of Artificial Sequence Note = Synthetic Construct
ggaaaugcaagagaaacugtt

SEQ ID NO: 8 - DNA, 21 bp, Artificial Sequence
  Description of Artificial Sequence Note = Synthetic Construct
ttccuuuacguucucuuugac

SEQ ID NO: 9 - DNA, 21 bp, Artificial Sequence
  Description of Artificial Sequence Note = Synthetic Construct
ggaaaugcaagagaaccugtt

SEQ ID NO: 10 - DNA, 21 bp, Artificial Sequence
  Description of Artificial Sequence Note = Synthetic Construct
ttccuuuacguucucuungac

SEQ ID NO: 11 - DNA, 22 bp, Artificial Sequence
  Description of Artificial Sequence Note = Synthetic Construct
ggaaaugcccagagaaacugtt

SEQ ID NO: 12 - DNA, 21 bp, Artificial Sequence
  Description of Artificial Sequence Note = Synthetic Construct
ttccuuuacgnucucuuugac

SEQ ID NO: 13 - DNA, 21 bp, Artificial Sequence
  Description of Artificial Sequence Note = Synthetic Construct
ggacaugcaagagaaacugtt

SEQ ID NO: 14 - DNA, 21 bp, Artificial Sequence
  Description of Artificial Sequence Note = Synthetic Construct
ttccunuacguucucuuugac

SEQ ID NO: 15 - DNA, 21 bp, Artificial Sequence
  Description of Artificial Sequence Note = Synthetic Construct
cagnuucucungcaunucctt

SEQ ID NO: 16 - DNA, 51 bp, Artificial Sequence
  Description of Artificial Sequence Note = Synthetic Construct
atcgctcgaggcacaggaaatgcaagagaaactgcagaagcggccgctggc

SEQ ID NO: 17 - DNA, 51 bp, Artificial Sequence
  Description of Artificial Sequence Note = Synthetic Construct
tagcgagctccgtgtcgtttacgttctctttgacgtcttcgccggcgaccg

SEQ ID NO: 18 - DNA, 21 bp, Artificial Sequence
  Description of Artificial Sequence Note = Synthetic Construct
ggaaaugcaagagaaacugtt

SEQ ID NO: 19 - DNA, 21 bp, Artificial Sequence
  Description of Artificial Sequence Note = Synthetic Construct
ttccuuuacguucucuuugac

SEQ ID NO: 20 - DNA, 21 bp, Artificial Sequence
  Description of Artificial Sequence Note = Synthetic Construct
ggaaaugcaagagaaacugtt

SEQ ID NO: 21 - DNA, 21 bp, Artificial Sequence
  Description of Artificial Sequence Note = Synthetic Construct
ttccuuuacguucucuuugac

SEQ ID NO: 22 - DNA, 21 bp, Artificial Sequence
  Description of Artificial Sequence Note = Synthetic Construct
ggacaugcaagagaaacugtt

SEQ ID NO: 23 - DNA, 21 bp, Artificial Sequence
  Description of Artificial Sequence Note = Synthetic Construct
ttccunuacguucucuuugac

SEQ ID NO: 24 - DNA, 21 bp, Artificial Sequence
  Description of Artificial Sequence Note = Synthetic Construct
ggaaaugccagagaaacugtt

SEQ ID NO: 25 - DNA, 21 bp, Artificial Sequence
  Description of Artificial Sequence Note = Synthetic Construct
ttccuuuacgnucucuuugac

SEQ ID NO: 26 - DNA, 21 bp, Artificial Sequence
  Description of Artificial Sequence Note = Synthetic Construct
ggaaaugcaagagaaccugtt

SEQ ID NO: 27 - DNA, 21 bp, Artificial Sequence
  Description of Artificial Sequence Note = Synthetic Construct
ttccuuuacguucucuungac

SEQ ID NO: 28 - DNA, 21 bp, Artificial Sequence
  Description of Artificial Sequence Note = Synthetic Construct
ggaaaugcaagagaaacugtt

SEQ ID NO: 29 - DNA, 21 bp, Artificial Sequence
  Description of Artificial Sequence Note = Synthetic Construct
ttccuuuacguucucuuugac

SEQ ID NO: 30 - DNA, 21 bp, Artificial Sequence
  Description of Artificial Sequence Note = Synthetic Construct
ggacaugcaagagaaacugtt

SEQ ID NO: 31 - DNA, 21 bp, Artificial Sequence
  Description of Artificial Sequence Note = Synthetic Construct
ttccunuacguucucuuugac

SEQ ID NO: 32 - DNA, 21 bp, Artificial Sequence
  Description of Artificial Sequence Note = Synthetic Construct
ggaaaugccagagaaacugtt

SEQ ID NO: 33 - DNA, 21 bp, Artificial Sequence
  Description of Artificial Sequence Note = Synthetic Construct
ttccuuuacgnucucuuugac

SEQ ID NO: 34 - DNA, 21 bp, Artificial Sequence
  Description of Artificial Sequence Note = Synthetic Construct
ggaaaugcaagagaaccugtt

SEQ ID NO: 35 - DNA, 21 bp, Artificial Sequence
  Description of Artificial Sequence Note = Synthetic Construct
ttccuuuacguucucuungac

SEQ ID NO: 36 - DNA, 21 bp, Artificial Sequence
  Description of Artificial Sequence Note = Synthetic Construct
ggaaaugcaagagaaacugtt

SEQ ID NO: 37 - DNA, 21 bp, Artificial Sequence
  Description of Artificial Sequence Note = Synthetic Construct
ttccuuuacguucucuuugac

SEQ ID NO: 38 - DNA, 21 bp, Artificial Sequence
  Description of Artificial Sequence Note = Synthetic Construct
ggacaugcaagagaaacugtt

SEQ ID NO: 39 - DNA, 21 bp, Artificial Sequence
  Description of Artificial Sequence Note = Synthetic Construct
ttccunuacguucucuuugac

SEQ ID NO: 40 - DNA, 21 bp, Artificial Sequence
  Description of Artificial Sequence Note = Synthetic Construct
ggaaaugccagagaaacugtt

SEQ ID NO: 41 - DNA, 21 bp, Artificial Sequence
  Description of Artificial Sequence Note = Synthetic Construct
ttccuuuacgnucucuuugac

SEQ ID NO: 42 - DNA, 21 bp, Artificial Sequence
  Description of Artificial Sequence Note = Synthetic Construct
ggaaaugcaagagaaccugtt

SEQ ID NO: 43 - DNA, 21 bp, Artificial Sequence
  Description of Artificial Sequence Note = Synthetic Construct
ttccuuuacguucucuungac

SEQ ID NO: 44 - DNA, 21 bp, Artificial Sequence
  Description of Artificial Sequence Note = Synthetic Construct
ggaaaugcaagagaaacugtt

SEQ ID NO: 45 - DNA, 21 bp, Artificial Sequence
  Description of Artificial Sequence Note = Synthetic Construct
ttccuuuacguucucuuugac

SEQ ID NO: 46 - DNA, 21 bp, Artificial Sequence
  Description of Artificial Sequence Note = Synthetic Construct
ttccuuuacgnucucuungac

SEQ ID NO: 47 - DNA, 21 bp, Artificial Sequence
  Description of Artificial Sequence Note = Synthetic Construct
ttccunuacgnucucuuugac

SEQ ID NO: 48 - DNA, 21 bp, Artificial Sequence
  Description of Artificial Sequence Note = Synthetic Construct
ttccunuacguucucuungac

SEQ ID NO: 49 - DNA, 21 bp, Artificial Sequence
  Description of Artificial Sequence Note = Synthetic Construct
ttccunuacgnucucuungac

SEQ ID NO: 50 - DNA, 21 bp, Artificial Sequence
  Description of Artificial Sequence Note = Synthetic Construct
ggaaaugcaagagaaacugtt

SEQ ID NO: 51 - DNA, 21 bp, Artificial Sequence
  Description of Artificial Sequence Note = Synthetic Construct
ttccuuuacguucucuuugac

SEQ ID NO: 52 - DNA, 21 bp, Artificial Sequence
  Description of Artificial Sequence Note = Synthetic Construct
ttccuuuacgnucucuungac

SEQ ID NO: 53 - DNA, 21 bp, Artificial Sequence
  Description of Artificial Sequence Note = Synthetic Construct
ttccunuacgnucucuuugac

SEQ ID NO: 54 - DNA, 21 bp, Artificial Sequence
  Description of Artificial Sequence Note = Synthetic Construct
ttccunuacguucucuungac

SEQ ID NO: 55 - DNA, 21 bp, Artificial Sequence
  Description of Artificial Sequence Note = Synthetic Construct
ttccunuacgnucucuungac